(19) United States
(12) Patent Application Publication
(10) Pub. No.: US 2003/0114789 A1
(43) Pub. Date: Jun. 19, 2003

(54) Needleless hypodermic injection device

(76) Inventors: Hans-Peter Haar, Wiesloch (DE); Hans List, Hesseneck (DE); Otto Fuerst, Viernheim (DE); Markus Mattern, Heppenheim (DE); George Bevan Kirby Meacham, Shaker Heights, OH (US)

Correspondence Address: HOFFMANN-LA ROCHE INC., PATENT LAW DEPARTMENT, 340 KINGSLAND STREET, NUTLEY, NJ, 07110

(21) Appl. No.: 10/314,535

(22) Filed: Dec. 9, 2002

Related U.S. Application Data
(60) Provisional application No. 60/340,681, filed on Dec. 14, 2001.

(51) Int. Cl. A61M 5/30

(52) U.S. Cl. 604/69

(57) ABSTRACT

A needleless hypodermic injector device comprises pyrotechnical means for generating gas sufficient for injecting a medication. The device includes a housing with a first chamber which contains a medication unit storing liquid medication to be injected and a second chamber which contains a propellant container, a propellant within the propellant container, and an igniter. The medication unit has a deformable first region and a second region with an injection outlet in liquid communication the first region. The first chamber of the housing includes two zones, a first zone containing the medication unit and a second zone which is in communication with the second chamber, so that upon ignition of the propellant in the second chamber gas generated thereby expands into the second zone of the first chamber, deforming the first region of the medication unit thereby causing ejection of the liquid medication through the ejection outlet.

### PRIORITY TO PROVISIONAL APPLICATION(S) UNDER 35 U.S.C. §119(E)

This application claims priority under 35 U.S.C §119(e) of Provisional Application Serial No. 60/340,681, filed Dec. 14, 2001.

### FIELD OF THE INVENTION

The invention is related to device for performing a needleless hypodermic injection of a liquid medication contained in a medication unit within the device.

The invention concerns in particular a needleless injection device which includes pyrotechnical means for generating within the device a predetermined pressure value necessary for injecting the medication.

### BACKGROUND OF THE INVENTION

International Patent Application WO 98/31409 describes an hypodermic injection apparatus which comprises a body wherein a medication unit containing an amount of a medication and an activatable gas generator are arranged. Pressure generated by activation of the gas generator is applied on a deformable part of the medication unit in order to eject the medication through an outlet of the medication unit. The gas generator comprises a propellant container which contains a propellant and associated ignition means for igniting the propellant and thereby activate the gas generator. The body and the mechanical structure of the apparatus have therefore to be strong enough in order to withstand the pressure generated within the apparatus by ignition of the propellant virtually infinite number of times. To meet these requirements the device has to be constructed from high strength material with the associated volume and weight. And also training in using those systems is needed.

### SUMMARY OF THE INVENTION

The instant invention is based on the discovery that a pyrotechnically driven injection device which has to withstand only one application can be constructed primarily of lightweight and low cost material.

A first aim of the invention is to provide a device of the above mentioned kind having technical features which eliminate the risk of accidental rupture of the housing of the device caused by the pressure peak which develops within the housing when a propellant is ignited within the housing in order to generate the pressure necessary to effect an injection, and which thereby provides highest security of the user against being injured due to such an accidental rupture of the housing.

A second aim of the invention is to provide a device of the above mentioned kind which in addition ensures that injections are easily and reliably performed even by a person which has received only little instruction or training.

A third aim of the invention is to provide a device of the above mentioned kind which makes use of technically relatively simple parts and which can be manufactured by simple manufacturing steps so that manufacturing cost of the device is relatively low and therefore the use of the device is economically competitive compared with use of conventional devices for performing needle injections. Under the aspects just mentioned, a particular aim of the invention is to provide an injector device the cost of which is so low that its use as a disposable or single use device is justified.

A fourth aim of the invention is to provide a design of the nozzle which is part of the medication unit of an injector device according to the invention which contributes to achieve the aim of enabling the performance of effective and reliable injections and all above mentioned aims.

According to a first aspect of the invention the above mentioned first to third aims are achieved by means of a device for performing a needleless hypodermic injection of a liquid medication contained in a medication unit within the device, said device including pyrotechnical means for generating within the device a predetermined pressure value necessary for injecting the medication, said device comprising

(a) a housing which is so configured and dimensioned that it is adapted to withstand or uptake alone, i.e. by itself, said predetermined internal pressure value

(b) a first chamber within said housing, said first chamber containing a medication unit configured and dimensioned to store a volume of liquid medication to be injected, said medication unit having a first region and a second region that are in liquid communication with each other, said first region being deformable and said second region having an ejection outlet, and

(c) a second chamber within said housing, said second chamber containing a propellant container, a predetermined amount of a propellant within said propellant container, and ignition means for igniting said propellant, said first chamber comprising two zones, a first zone containing said medication unit and a second zone which is in communication with said second chamber, so that upon ignition of the propellant in the second chamber gas generated thereby expands into said second zone of said first chamber, exerts pressure on and deforms said deformable first region of said medication unit and thereby causes ejection of said liquid medication through said ejection outlet.

According to a second aspect of the invention the above mentioned first to third aims are achieved by means of a device for injecting a liquid medication comprising

(a) a nozzle body,

(b) a rigid housing,

said housing having

a first open end adapted to receive and be connected with the nozzle body and a second closed end,

the interior of said housing defining a chamber which extends between said open end and said closed end of the housing, said chamber being adapted to receive

a first deformable diaphragm which together with a cavity of said nozzle body forms a medication chamber suitable for receiving a predetermined amount of a medication, and

a second deformable diaphragm a portion of which extends around a portion of said first deformable diaphragm, said second deformable diaphragm and said housing forming together a chamber for receiving a propellant and means for igniting said propellant,

said nozzle body having at its outer end an orifice which is the outlet of a channel for loading a liquid medication into the medication chamber and for ejecting said medication out of said chamber when a gas pressure generated by ignition of said propellant is applied to said second deformable diaphragm and thereby to said first deformable diaphragm.

According to a third aspect of the invention the above mentioned first to third aims are achieved by means of a device for performing a needleless hypodermic injection of a liquid medication contained in a medication unit within the device, said device including pyrotechnical means for generating within the device a predetermined pressure value necessary for injecting the medication, said device comprising

(a) a medication unit for storing a volume of liquid medication to be injected, said medication unit having a first region and a second region that are in liquid communication with each other, said first region being deformable and said second region having an ejection outlet,

(b) a first rigid housing part having a first zone for receiving said medication unit,

(c) a second rigid housing part for receiving and/or carrying said pyrotechnical means,

said first and second housing parts being connectable with each other and defining a single chamber, and

(d) a deformable barrier arranged within said single chamber and dividing said chamber in two zones, a first zone wherein said medication unit is located and a second zone where said propellant is located, pressure generated by combustion of said propellant being directly applied to said deformable barrier and thereby to said deformable region of said medication unit for ejecting said medication through said ejection outlet of said second region of said medication unit.

According to a fourth aspect of the invention the above mentioned fourth aim is achieved by means of a nozzle which is part of a medication unit of a device according to the invention, said nozzle having a body having a longitudinal axis which is also a rotation symmetry axis of said body, said body comprising an injection channel having a symmetry axis that coincides with said symmetry axis of said body, said injection channel having at a first end thereof an inlet connectable to a medication container, said injection channel having at a second end thereof opposite to said first end an outlet for delivering medication ejected through said injection channel, and said body having a neck portion that ends in a first end which forms a contact surface with the skin at the injection site, a basis portion that ends in a second end opposite to said first end of said body, and an intermediate portion that extends between said neck portion and said basis portion.

The main advantages obtained with a device according to the invention are as follows:

The design of the gas pressure generator is optimized for generating the gas pressure required to perform a needleless hypodermic injection with a very small amount of propellant. This feature makes it possible to use simple and cost effective structures for manufacturing the device. This is achieved in particular by using a pyrotechnic gas generator that is as small as possible, and has only very little heat loss.

Protection of the user against possible injury in case of any type of failure of the device due to the inner pressure peak during the injection process is ensured by the structure of the device according to the invention, which includes a housing which is so configured and dimensioned that it is adapted to withstand an internal pressure higher than the normal injection pressure without yielding. A preferred embodiment comprises in addition a protective envelope of the housing of the device, the envelope having the shape of a tubular layer of a tough elastic material, e.g. polyethylene.

Very reliable operation of a device according to the invention is ensured by the provision of features which only allow the performance of an injection when some well defined conditions are satisfied.

A particularly convenient design of the nozzle which is part of the medication unit of an injector device according to the invention contributes to achieve the above mentioned aim of the invention.

Low manufacturing cost of a device according to the invention is achieved by the choice of suitable and low cost materials and by a device structure which optimally meets the operation, reliability and safety requirements, and which comprises a highly efficient gas generator which has a simple structure. Therefore such a device is suitable for use as a disposable or single use injector device.

The embodiments of the invention described hereinafter have advantageous features and characteristics which are described and explained more closely below with reference to the representation of the drawings.

### BRIEF DESCRIPTION OF THE DRAWINGS

The subject invention will now be described in terms of its preferred embodiments with reference to the accompanying drawings. These embodiments are set forth to aid the understanding of the invention, but are not to be construed as limiting.

FIG. 1 shows a schematic cross-sectional view of a first embodiment of a needleless injector module 11 according to the invention and comprising an intermediate support and a rear plug integrally formed with each other.

FIG. 2a shows a perspective cross-sectional view of the device shown by FIG. 1.

FIG. 2b shows a perspective cross-sectional and exploded view of the components of the module shown by FIG. 1.

FIG. 3a shows a module having the structure shown by FIG. 1 and shows on the right side a portion enclosed by a circle IIIb which comprises a first embodiment of a controlled bleed vent using e.g. a paper gasket.

FIG. 3b is an enlarged view of the portion IIIb shown by FIG. 3a.

FIG. 4a shows a module having the structure shown by FIG. 1 and shows on the right side a portion enclosed by a circle IVb which comprises a second embodiment of a controlled bleed vent using e.g. wax as sealing means.

FIG. 4b is an enlarged view of the portion IVb shown by FIG. 4a with the wax as sealing means before an injection is performed with the module.

FIG. 4c is an enlarged view of the portion IVb shown by FIG. 4a after wax melts and thereby opens a vent after an injection is performed with the module.

FIG. 5a is a cross-sectional view of a first propellant container which can be part of the module shown by FIG. 1.

FIG. 5b is a front view of a cap of the propellant container shown by FIG. 5a.

FIG. 5c is a cross-sectional view of the cap of the propellant container shown by FIG. 5a.

FIG. 6 is a cross-sectional view of a second propellant container which can be a part of the module shown by FIG. 1, a part of the volume of this container being filled by aerogel.

FIG. 7a is a cross-sectional view of a third propellant container which can be a part of the module shown by FIG. 1, a part of the volume of this container being filled by a pocket filled with air before ignition of the propellant.

FIG. 7b is a cross-sectional view showing the third propellant container shown by FIG. 7a during the ignition process.

FIG. 8 shows a schematic cross-sectional view of a second embodiment of a needleless injection module according to the invention and comprising as separate parts an intermediate support and a rear plug.

FIG. 9 shows an exploded cross-sectional view of the module shown by FIG. 8.

FIG. 10a shows a schematic cross-sectional view of a third embodiment of a needleless injection module according to the invention, this embodiment having a deformable zone and an O-ring seal which together form overpressure control means.

FIG. 10b shows a perspective cross-sectional view of the components of the module shown by FIG. 10a.

FIG. 10c shows a perspective cross-sectional and exploded view of the components of the module shown by FIG. 10a.

FIG. 11 shows a schematic cross-sectional view of the third embodiment shown by FIG. 10 in combination with mechanical impact ignition means.

FIG. 12 shows an enlarged view of an end part of the module shown in FIG. 11.

FIG. 13 shows a typical pressure vs. time diagram of the pressure exerted on the medication container when an injection is effected with a injector module according to the invention.

FIG. 14 shows a schematic cross-sectional view of an injector device according to the invention comprising a battery and switch mechanism for ignition.

FIG. 15 shows a schematic cross-sectional view of an injector device according to the invention comprising a battery, and a switch mechanism for ignition that includes object sensing means.

FIG. 16a shows a schematic cross-sectional view of an injector device according to the invention comprising a battery and switch mechanism for ignition that includes an interlocking object sensor function, this device being shown in a first state.

FIG. 16b shows a schematic cross-sectional view of the device shown by FIG. 16a in a second state.

FIG. 16c shows a schematic cross-sectional view of the device shown by FIG. 16a in a third state.

FIG. 17 shows a perspective exploded view of components of the object sensor interlock shown by FIGS. 16a to 16c.

FIGS. 18a to 18c show different views of a first preferred embodiment of a nozzle of the medication unit which is part of an injector module according to the invention,

FIGS. 19a to 19c show different views of a second preferred embodiment of a nozzle of the medication unit which is part of an injector module according to the invention.

FIG. 20 shows a schematic cross-sectional view of a fourth embodiment of a needleless injector module according to the invention.

FIG. 21 shows a schematic cross-sectional view of a fifth embodiment of a needleless injector module according to the invention.

FIG. 22 shows a schematic cross-sectional view of a sixth embodiment of a needleless injector module according to the invention.

FIG. 23 shows a schematic cross-sectional view of a seventh embodiment of a needleless injector module according to the invention.

FIG. 24 shows a one-piece propellant pellet.

FIGS. 25-27 show several arrays comprising several one-piece propellant pellets.

### DETAILED DESCRIPTION OF PREFERRED EMBODIMENTS

A first, a second and a third embodiment of an injector module according to the invention, called generically injector module 11, are first described hereinafter. This description is followed by a description of particular aspects and uses of such an injector module, including a description of an injection device comprising such an injector module.

### FIRST EMBODIMENT OF A DEVICE ACCORDING TO THE INVENTION

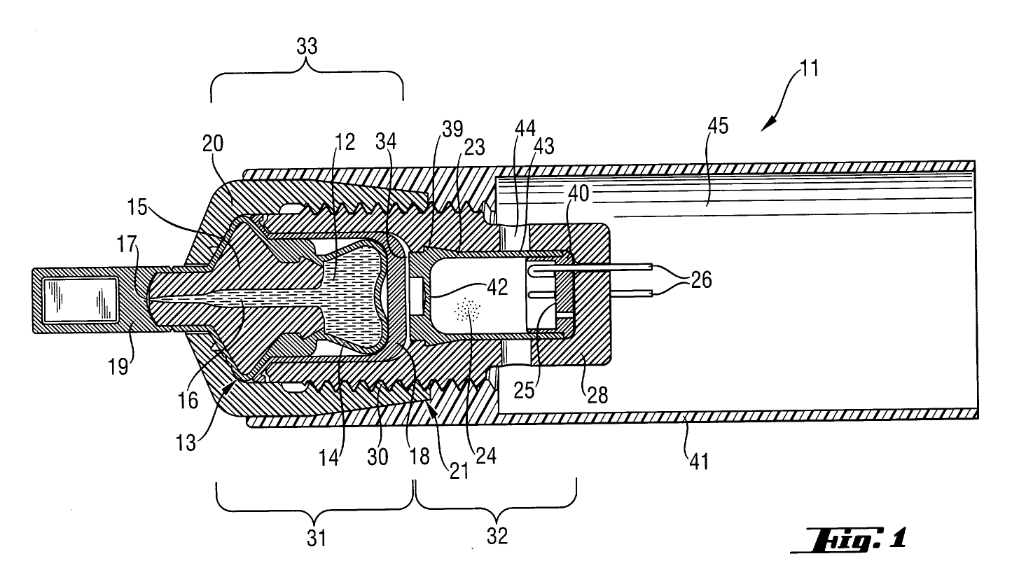

A first embodiment of a single use injector module 11 according to the invention is described hereinafter with reference to FIGS. 1 to 7.

As shown by FIG. 1 a single use injector module 11 according to the invention comprises components described hereinafter.

Single use injector module 11 comprises a housing 21 formed by the assembly of a pressure cell 20 and a support member 28 which is closed at one end and has also the function of a rear plug for pressure cell 20.

Pressure cell 20 and a support member 28 have threads which match with each other and are thus be connected with each other by a screw connection 30.

Housing 21 is so configured and dimensioned that as a whole is adapted to withstand an internal pressure which is higher than the normal injection pressure without yielding.

Housing 21 is made preferably of a thermoplastic plastic material. A suitable housing material can be chosen e.g. from commercially available polyesters or polycarbonates taking in particular into account the mechanical properties the housing should have.

Further criteria for choosing the housing material are that it should allow relatively large dimension tolerances, that the housing should keep its original shape in order to maintain a constant volume of the housing and to be suitable for being connected to other components simply by a snap connection, and that the housing material should be physiologically suitable for the intended use.

In order to ensure a safe operation of injector module 11 even if pressure within the housing accidentally exceeds a predetermined normal injection pressure, the material, shape and dimensions of the housing 21 are preferably so chosen that it has a predetermined failure zone where the housing breaks if an unduly high pressure peak arises within the housing, so as to allow gas escape from the housing in a controlled way. In a preferred embodiment housing 21 has a zone of reduced thickness (not shown in FIG. 1), which bursts so as to allow gas escape in a controlled way if an unduly high pressure peak arises within the housing, e.g. when that pressure exceeds a predetermined value.

The interior of housing 21 comprises a first chamber 31 and a second chamber 32, which are defined for instance by respective cavities of a support member 28.

A medication unit 13 is arranged within first chamber 31. A volume of liquid to be injected is stored in medication unit 13. In preferred embodiments, the amount of this volume is in a range going from about 50 to 1000 microliters. Specific examples of this amount are e.g. 200 or 500 microliters.

Medication unit 13 is a sealed medication module which comprises a nozzle body 15 and a flexible container wall 14 that hermetically encloses a portion of the nozzle and forms a reservoir 12 for a liquid medication stored in sealed medication unit 13. Wall 14 is deformable and collapsible.

Medication unit 13 thus comprises a first region and a second region that are in liquid communication with each other. The first region is deformable and comprises the reservoir enclosed by flexible wall 14. The second region of medication unit 13 comprises nozzle body 15 which has a fluid channel 16 that ends in an orifice 17 which serves as a liquid jet outlet through which liquid to be injected is ejected when an injection is performed with injector module 11. Medication unit 13 is made of suitable construction materials, e.g. polyethylene and polypropylene, which are suitable for storing medications including sensitive protein drugs.

A part of container wall 14 forms a break-off protective cap 19 that covers a jet orifice 17 of nozzle body 15. Cap 19 is removed by the user just prior to use of injector module 11.

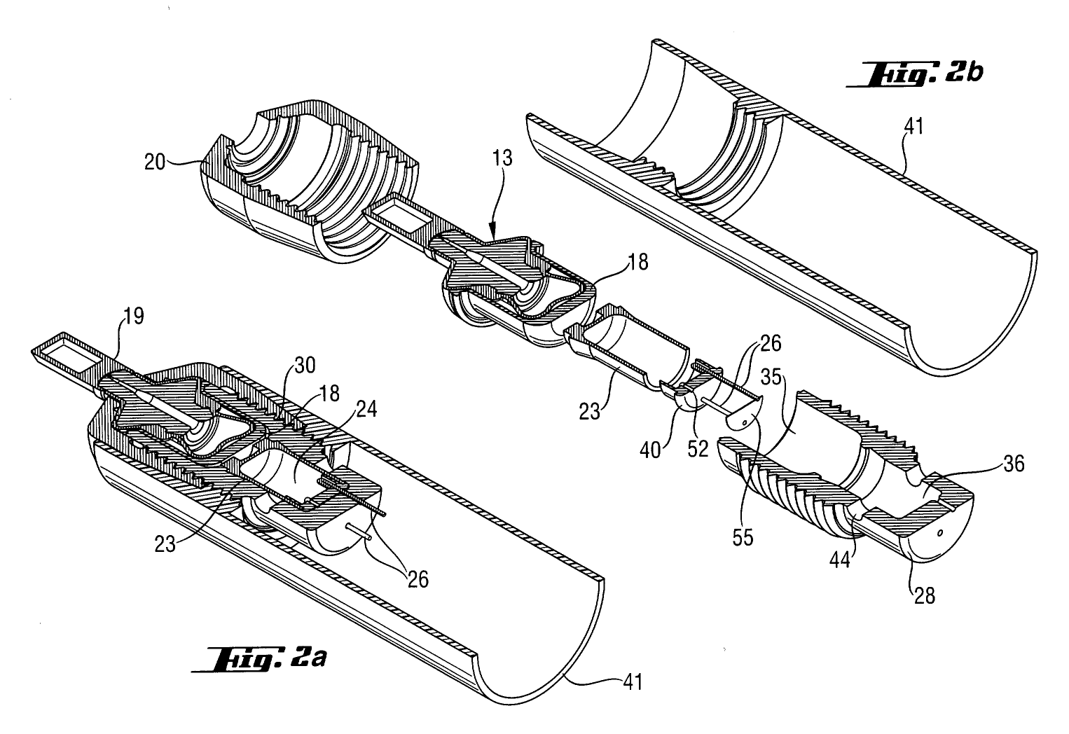

A propellant container 23 is arranged within second chamber 32 of housing 21. Propellant container 23 contains a predetermined amount of a propellant 24. Propellant container 23 is closed by a lid 40 which carries e.g. ignition pins for electrically igniting propellant 24. When assembling injector module 11, propellant container 23 is loaded with propellant 24, e.g. in powder form, propellant container 23 is then closed by lid 40, and the so closed propellant container 23 is fitted within support member 28. As shown by FIGS. 2a and 2b, lid 40 is disposed within support member 28. Support member 28 thus receives medication unit 13 and propellant container 23. Within the scope of the instant invention a propellant is a pyrotechnic fuel which mainly contributes to the delivery of thermal energy and gas production of a pyrotechnic system and an ignition material is a pyrotechnic material used in a pyrotechnic initiator for initiating combustion of a propellant.

First chamber 31 comprises two zones, a first zone 33 which contains medication unit 13 and a second zone 34 which is located between medication unit 13 and second chamber 32.First chamber 31 is in communication with second chamber 32 so that upon ignition of propellant 24 in propellant container 23 located within second chamber 32, gas thereby generated expands into second zone 34 of first chamber 31, exerts pressure on and deforms deformable wall 14 of the first region of medication unit 13 and thereby causes ejection of the liquid medication through channel 16 and orifice 17.

In a preferred embodiment an elastic barrier 18 divides the first zone 33 from the second zone 34. The elastic barrier is made e.g. of silicon rubber, and can be reinforced e.g. with woven polyamide fibers, preferably polyaramide fibers.

Support member 28 is made preferably of a rigid, plastic material which does rather break than yield when subject to mechanical stress. Support member 28 is made e.g. of thermoplastic polyester or a polycarbonate having the above mentioned properties.

As can be appreciated in particular from FIG. 2b, support member 28 has a first cavity 35 which defines part of the first chamber, a second cavity 36 which defines part of the second chamber.

In a preferred embodiment the free-volume comprised between medication unit 13 and the wall of propellant container 23 which faces medication unit 13 is much smaller than the volume of propellant container 23.

In a preferred embodiment of the single use injector module described with reference to FIGS. 1-4, as well as in preferred embodiments of all other single use injection modules described with reference to the other drawings attached to this specification, housing 21 is enveloped by a tubular layer 41 which is an outer shield of injector module 11. The thickness of this layer is e.g. about 0.4 millimeter.

Tubular layer 41 is preferably made of a stretchable or compliant material which is adapted to form an outer shield which protects the user of the injector module from exhaust gas that may leak from the housing and from splinters of the housing in the event that the housing would accidentally burst due to excessive internal pressure or material failure.

Tubular layer 41 is preferably made of a polymer (e.g. polyolefine, polyolefinic acid esters, polyurethanes), in particular of polyethylene, or of soft steel, or of soft aluminum.

Propellant 24 is e.g. a fine grain nitrocellulose based composition or another nitrocellulose based composition, or another propellant composition having similar properties or a mixture of propellant compositions.

The embodiment shown by FIGS. 1, 2a, 2b is characterized by a seal clamping geometry which eliminates gas leaks by achieving short stress paths that minimize undesirable deflection of the components under pressure. This seal clamp geometry is particularly important when the components of the injector module are made of plastic materials, because plastic is in general much more elastic (about 30 times) than e.g. aluminum.

The design shown by FIGS. 1, 2a, 2b makes it possible to considerably reduce deflections which would otherwise decrease system efficiency by increasing free volume, and which would cause distortions that would make more difficult to achieve proper sealing of the injector module.

Pressure shell 20 and support member 28 in combination form a housing 21 that is a pressure vessel that carries the axial and circumferential stresses generated by the internal pressure in housing 21. The short axial stress path in the embodiment shown by FIGS. 1, 2a, 2b is achieved by connecting pressure shell 20 to support member 28 by a screw connection 30 close to the nozzle end of the injector module. This results in a short unidirectional axial stress path between pressure shell 20 and support member 28. Circumferential stress is resisted by the double layer consisting of the engaged threaded sections of pressure shell 20 and support member 28. With this design, a pressure shell 20 made of polycarbonate or another suitable plastic may be used without excessive deflection of housing 20 despite the inherent elasticity of the plastic material of which its components are made.

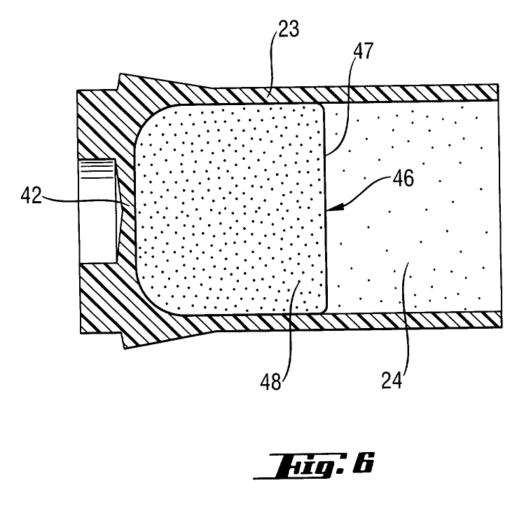

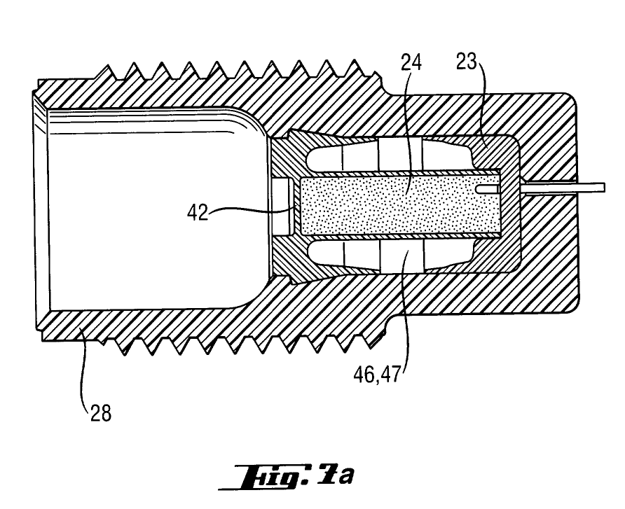

FIG. 5a shows a cross-sectional view of a first propellant container 23 which can be part of injector module 11 shown by FIG. 1. FIG. 5b shows a front view of a cap 40 of propellant container 23 shown by FIG. 5a. FIG. 5c is a cross-sectional view of cap 40.

As shown by FIG. 5a, a wall of propellant container 23 has preferably a zone 42 which has a reduced thickness. As shown by FIG. 1, this zone 42 lies between the interior of propellant container 23 and first chamber 31 shown e.g. in FIG. 1. Zone 42 is so configured and dimensioned that it bursts and thereby creates an opening when the pressure developed within propellant container 23 after ignition of propellant 24 reaches a predetermined value. In a preferred embodiment that predetermined pressure value, e.g. 100 bar, is lower than the normal injection pressure, e.g. 300 bar.

In preferred embodiments of a injector module according to the invention, propellant container 23 or at least the inner walls thereof are made of a plastic material which has a low thermal conductivity and therefore absorbs a very low amount of heat from the hot gas generated within container 23 by ignition of propellant 24, which does not show a significant chemical reaction with either the propellant or that hot gas. Such a plastic material is e.g. polyethylene or a plastic material having similar properties.

In a preferred embodiment, propellant container 23 has e.g. the structure shown by FIG. 6. In order to limit the amount of propellant 24 that can be introduced into the propellant container 23, a body 46 which contains air is introduced into propellant container 23 before filling it with propellant 24. Such a body can for instance be a pocket 47 containing aerogel material 48. Within the scope of the instant invention an aerogel is e.g. a fine silica based low weight powder which is suitable for being used as a filler within a mixture of other chemicals, e.g. in an ignition mixture. Aerogels are e.g. low weight polymeric bodies with a 3 dimensional network, produced starting from a gel by evaporating solvent (mostly water) under appropriate conditions. The density of such aerogels is only about 3 times the density of air. Within the scope of the instant invention an aerogel can also be a solid material formed of the above mentioned aerogel in powder form that can be handled as a block in order to fill a certain volume.

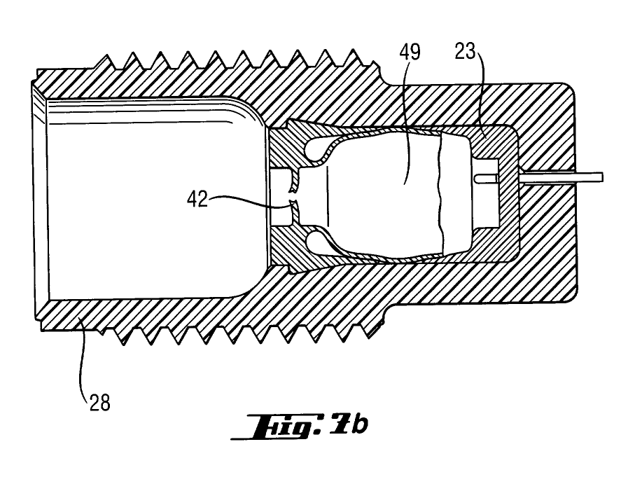

FIG. 7a shows a variant of the propellant container shown by FIG. 6. In this variant the space available for propellant 24 within propellant container 23 is limited by a body 46, 47 which surrounds a central elongated part of propellant container 23. That body can also be a kind of pocket which contains e.g. air or an aerogel material. When propellant 24 is ignited, the latter body is burned out. As shown by FIG. 7b, when this happens the volume 49 available within propellant container 23 for the gas generated is larger than the volume available for propellant 24 before ignition thereof.

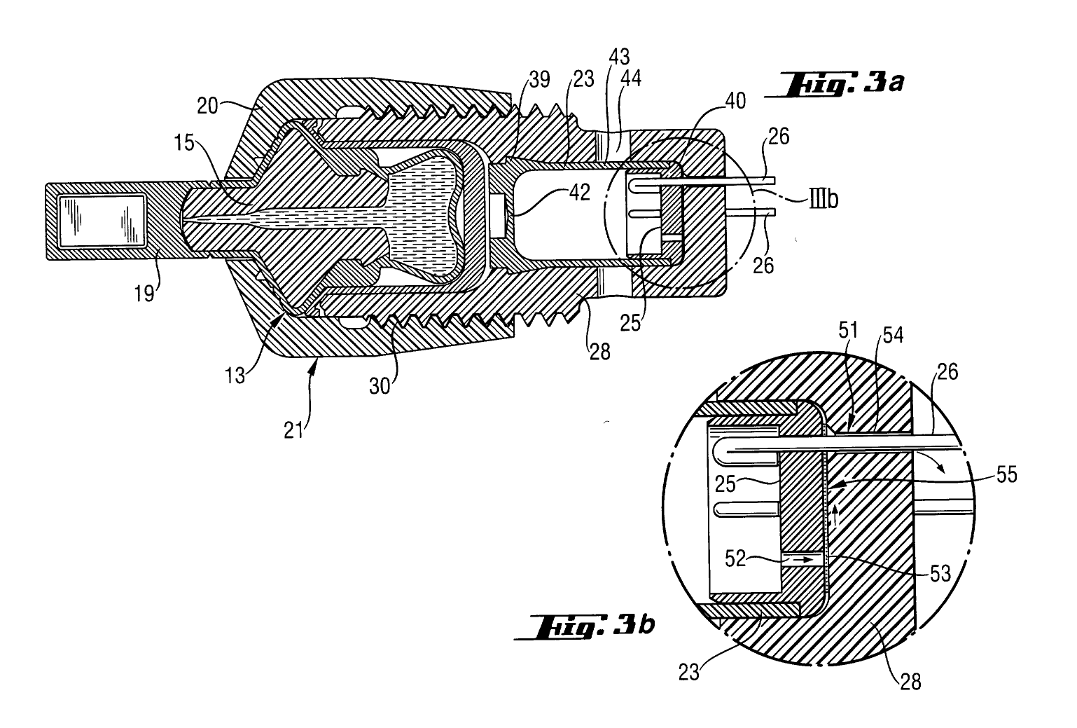

FIG. 3a shows a injector module having the structure of the injector module shown by FIG. 1.

As can be appreciated from FIG. 1 and also from FIG. 3a, the embodiment represented therein is characterized in that the lateral wall of propellant container 23 has at least one safety rupture zone 43 and that the housing of the propellant container 23 has a corresponding safety vent hole 44.

In connection with the safety means just described it is important to note that propellant container 23 is made of a material which has a much lower strength than the material of support member 28 wherein propellant container 23 is lodged. Propellant container 23 has walls which are so thin that they get torn at a pressure just above the predetermined normal injection pressure which has a maximum value of e.g. 300 bar. Moreover the material of which propellant container 23 is made has a softening temperature which lies under the ignition point of the propellant. Therefore, if the injector module would happen to be subject to an unusually high environment temperature, e.g. if the injector module is unduly kept in a container exposed to sun light irradiation over a certain time, such an external heating would profoundly weaken propellant container 23 at temperatures below the propellant ignition temperature. Consequently, under the circumstances just described (device subject to unusually high environment temperature), the safety rupture zone 43 of propellant container 23 would fail at a very low pressure and vent the gas into an attenuation volume 45 inside tubular layer 41 shown in FIG. 1.

FIG. 3a shows on the right side a portion enclosed by a circle IIIb which comprises a first embodiment of a controlled bleed vent. FIG. 3b shows an enlarged view of that portion IIIb.

As shown by FIG. 3b, the embodiment represented in FIG. 3a is characterized in that it comprises a very narrow controlled bleed vent passage 51 that leads from the inside to the outside of housing 21 such that gas within the housing is vented to atmosphere. Passage 51 comprises e.g. a vent channel 52, a vent passage 53 and a vent exit 54 around an ignition pin 26. Passage 51 has preferably a flow resistance such that flow of gas through the passage is negligible during the injection period, but vents injector module 11 to atmospheric pressure after the injection period. It should be noted that the injection period is a very short period during which the medication unit is suddenly squeezed by the injection pressure generated by ignition of propellant 24 and liquid medication thereby ejected from medication unit 13 is injected through the skin of the patient.

In a preferred embodiment passage 51 leading from the inside to the outside of propellant container 23 includes a flow resistance element 55 such that flow is negligible during an injection period of about 50 milliseconds, but vents injector module 11 to atmospheric pressure within a time interval comprised between about 10 seconds and some minutes.

In a preferred embodiment, flow resistance element 55 is a gasket based on cellulose, e.g. a paper gasket, is inserted in at least one segment of passage 51 to form a controlled leak which vents the housing after a normal injection.

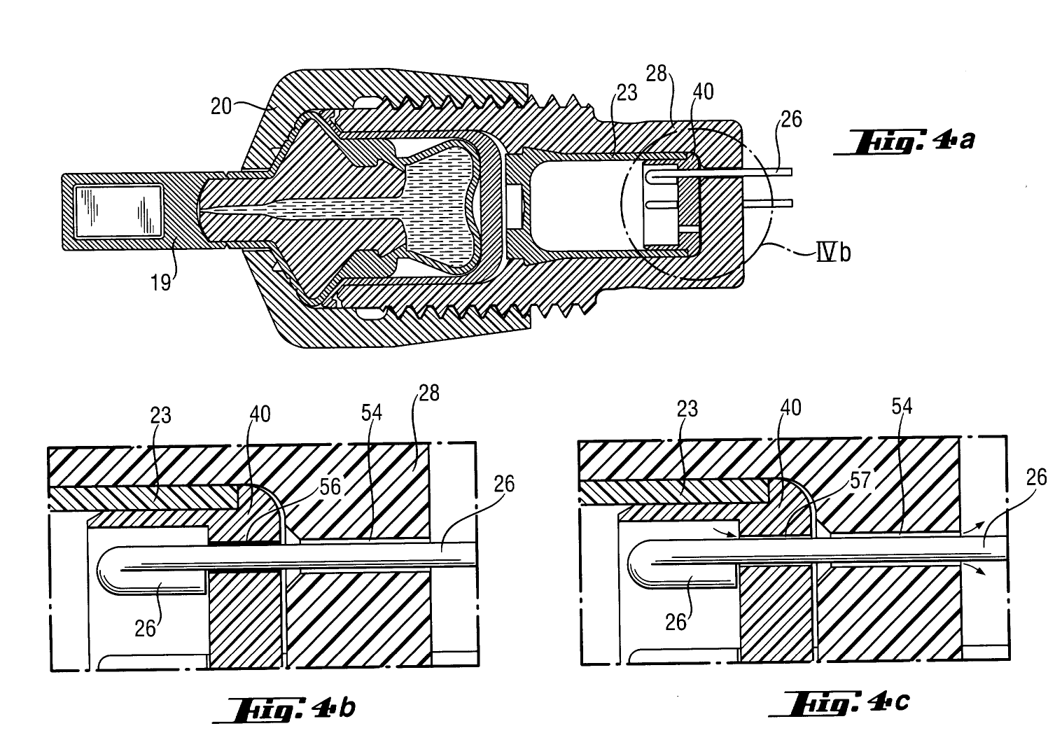

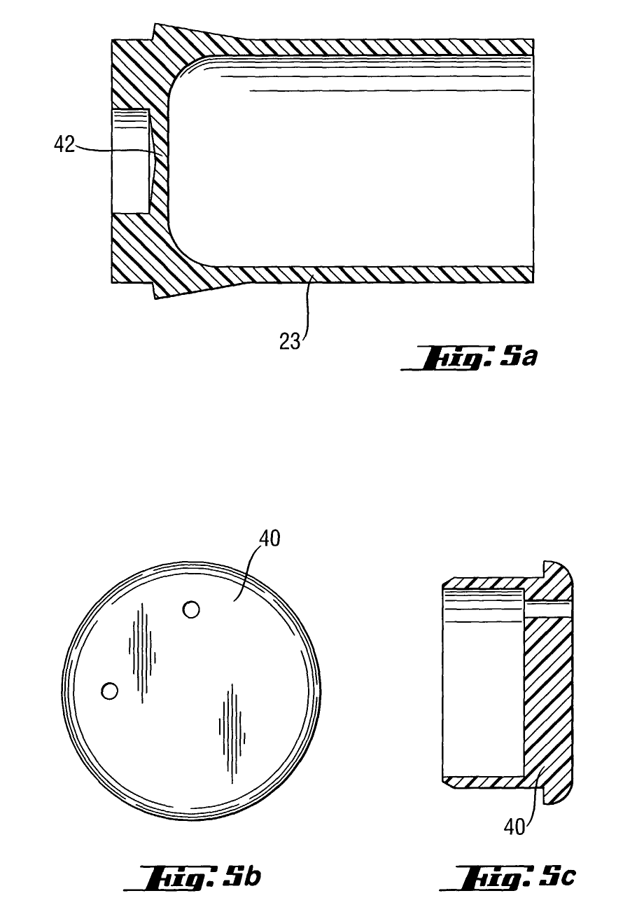

FIG. 4a shows a injector module 11 having the structure shown by FIG. 1 and shows on the right side a portion enclosed by a circle IVb which comprises a second embodiment of a controlled bleed vent using e.g. wax as sealing means. FIG. 4b is an enlarged view of the portion IVb shown by FIG. 4a with a wax layer 56 as sealing means before an injection is performed with injector module 11. FIG. 4c is an enlarged view of the portion IVb shown by FIG. 4a after wax layer 56 melts and thereby opens an annular clearance vent 57 after an injection is performed with injector module 11.

In the embodiment represented in FIGS. 4a to 4c a passage 57 formed around an electrically contacting ignition pin 26 contains a temperature sensitive substance such that flow through that passage 57 is blocked by the substance during the 50 millisecond injection period, and is later melted by heat generated by burning of propellant 24 and vents injector module 11 to atmospheric pressure. A temperature sensitive substance suitable for the latter purpose is e.g. a wax having a sharply defined melting point.

The embodiment described above with reference to FIGS. 1 to 4c has the following advantages:

The internal volume and surface area contacted by hot propellant gas are minimized, because the components that enter into contact with hot gas are made of materials such as polyethylene and polycarbonate with low transient heat absorption. This substantially increases the thermal efficiency of injector module 11 and thereby reduces the amount of energy needed to perform an injection and thereby the amount of propellant required for that purpose. The maximum energy content of injector module 11 is thus limited, and consequently the need for reinforcing the structure of injector module 11 with additional structure in order to handle overpressure events is reduced.

Pressure shell 20 and support member 28 are designed to minimize the stress path length, and thereby minimize the volumetric expansion even when relatively elastic materials such as polycarbonate are used. This also makes gas sealing easier, since the seal geometry changes less under pressure. Support member 28 is preferably made of plastic to reduce losses of heat generated by ignition of the propellant gas. Pressure shell 20 does not contact the gas, and may be made of any plastic or metal with sufficient strength and ductility.

Safety rupture zones are included in the structure of the injector module to vent gas from the inside of the structure in the event that the pressure rises significantly higher than needed for the injection. To protect the user the gas is vented into an attenuation volume 45 inside the polyethylene outer shield 41 to protect the user.

A controlled bleed vent reduces the internal pressure to atmospheric within a few seconds to a few minutes after the injection.

The number of components of injector module 11 is minimized, and all are designed for low-cost automated manufacture and assembly.

### SECOND EMBODIMENT OF A DEVICE ACCORDING TO THE INVENTION

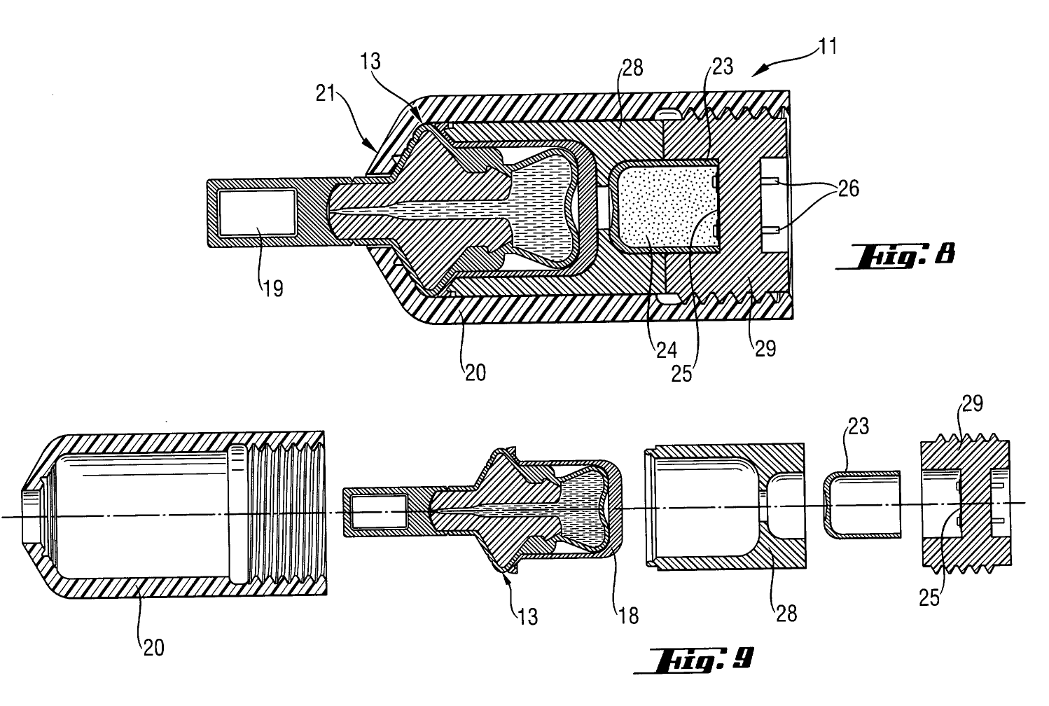

FIG. 8 shows a schematic cross-sectional view of a second embodiment of a needleless injector module according to the invention. FIG. 9 shows an exploded cross-sectional view of the injector module shown by FIG. 8.

This second embodiment has components which perform similar functions as in the first embodiment, but support member 28 and rear plug 29 are separate parts. This second embodiment is a viable product, it does however have longer stress paths than the first embodiment, and consequently has a higher volumetric expansion. Moreover sealing of this injector module 11 is more difficult if pressure shell 20 is made of plastic. With an aluminum pressure shell 20 volumetric expansion is lower, and a good sealing of the injector module is easier.

### THIRD EMBODIMENT OF A DEVICE ACCORDING TO THE INVENTION

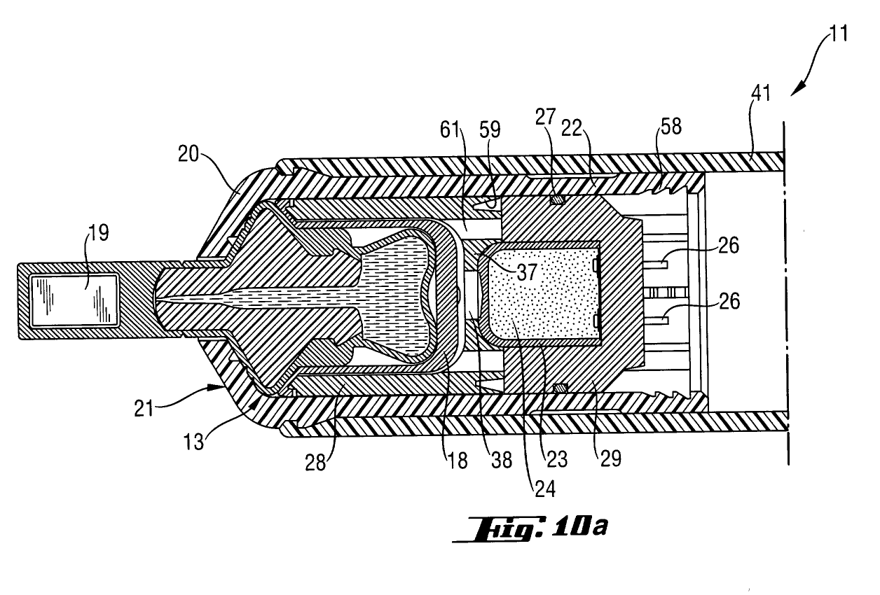

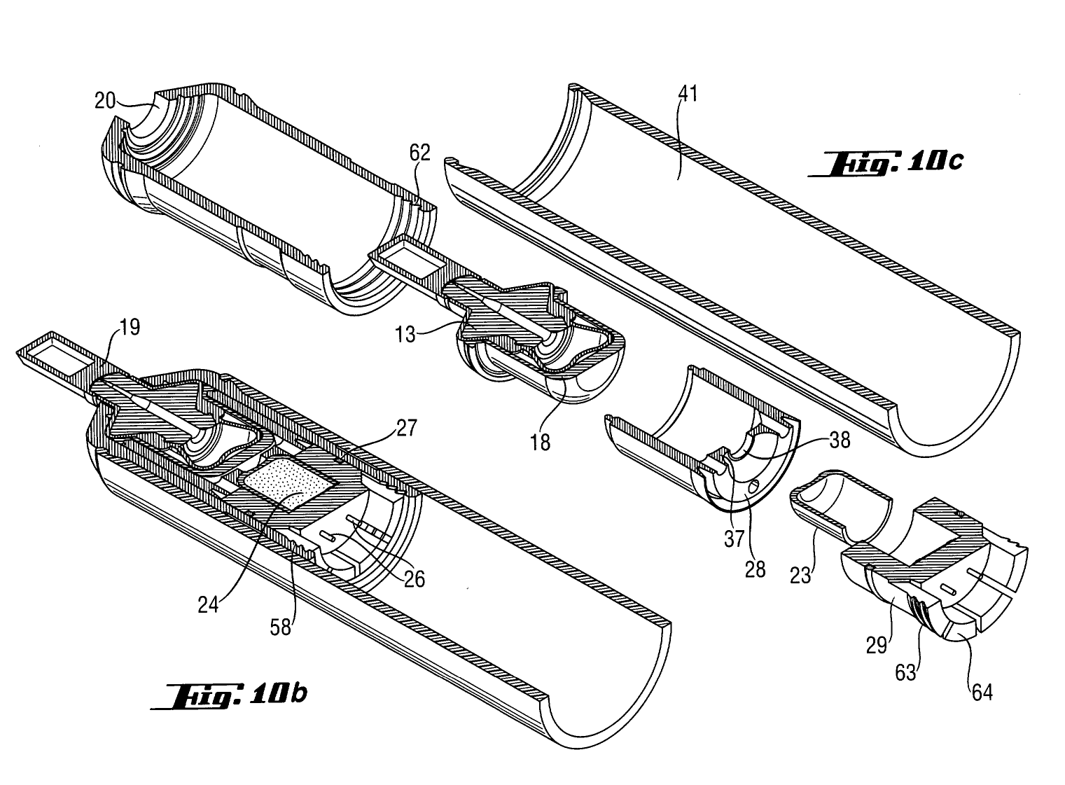

FIG. 10a shows a schematic cross-sectional view of a third embodiment of a needleless injector module 11 according to the invention. FIG. 10b shows and a perspective cross-sectional view of the components of the device shown by FIG. 10a. FIG. 10c shows a perspective cross-sectional and exploded view of the components of injector module 11 shown by FIG. 10a.

This third embodiment has a structure similar to the structure of the second embodiment shown by FIGS. 8 and 9, but has in addition a deformable zone 22 and an O-ring seal 27 which together operate as vent means in case that an unduly high pressure is generated within injector module 11.

As shown by FIG. 10a, in this third embodiment support member 28 fills the space comprised between the cavities 35 and 36 (shown in FIG. 2b) and housing 21, and housing 21 and rear plug 29 are connected with each other by means of a snap connection 58. For this purpose housing 21 and rear plug 29 have snap grooves 62 and 63 that match with each other. There is a lip seal 59 between support member 28 and pressure shell 20.

In this third embodiment the material, shape and dimensions of housing 21 are so chosen that housing 21 has at least one deformable zone 22 that rather yields than breaks in the event that the internal pressure reaches a predetermined level above the normal injection pressure, and thereby vents the housing and prevents rupture of housing 21. For this purpose housing 21 is e.g. operatively associated with means which allow venting of the housing under such circumstances. As shown by FIG. 1, housing 21 has e.g. a zone 22 of reduced thickness which cooperates with an O-ring 27 so as to allow gas escape in a controlled way if an unduly high pressure peak arises within housing 21, e.g. when that pressure exceeds a predetermined value. Such a housing thus has a wall having a zone of reduced structural strength which cooperates with sealing means adapted to yield so as to allow gas escape in a controlled way if an unduly high pressure peak arises within the housing. In other terms, in such an embodiment the assembly of housing 21 and of the components contained therein has at least one predetermined leakage zone at which a leak arises in the event that the internal pressure reaches a predetermined level above the normal injection pressure, and that leak vents housing 21 and prevents rupture thereof. In addition intermediate wall 37 of support member 28 preferably includes safety vent holes 61 shown in FIG. 10a. The venting means just described with reference to the embodiment shown by FIGS. 10a to 10c can also be part of the embodiment shown by FIGS. 8 and 9.

It should noted that in the third embodiment shown by FIGS. 10a, 10b, 10c when the structure of the injector module is subject to mechanical stress due to the pressure generated within the injector module by the pyrotechnical gas pressure generator, there is a long, bidirectional stress path leading from the nose of the pressure shell formed by housing 21 back to rear plug 29, and then forward through ignition plate 25 (shown in FIG. 10). Moreover, the circumferential pressure stress in the pressurized volume between support member 28 and pressure shell 20 forward of the O-ring seal acts with full force on pressure shell 20. Experiments have shown that the elasticity of the structure of injector module 11 in these regions causes a loss of part of the pressure generated by pyrotechnical gas pressure generator. Since a pressure shell 20 made e.g. of a suitable thermoplastic material is more deformable than a similar pressure shell made of aluminum, the pressure value generated by ignition of a given amount of propellant 24 contained in a plastic propellant container 23 is about 20 to 25% lower than the pressure value generated under similar conditions within an aluminum pressure shell.

### FOURTH EMBODIMENT OF A DEVICE ACCORDING TO THE INVENTION

FIG. 20 shows a schematic cross-sectional view of a fourth embodiment of a needleless injector module according to the invention.

This fourth embodiment has components which perform similar functions as in the second embodiment, but is characterized by the following features:

An aluminum pressure cell 20 contains all other components of the injector module.

A polyethylene propellant container 23 and an ignition plate 25 with a lip seal 116 are contained between an intermediate carrier 28 and a rear housing 29. This arrangement results in parts that are simpler to mold than the snap-fit polyethylene ignition container used in other embodiments described above.

Propellant container has a burst membrane 42a. This membrane is a zone of the wall of propellant container 23 which has a reduced thickness, which in contrast to burst membrane 42 of other embodiments described above is thin at the edges and thick in the middle. This shape of membrane 42a is advantageous, because when the membrane burst under a sudden rise of pressure in the propellant container 23, membrane 42a swings open like a door and the entire surface of membrane 42a is suddenly open and thereby the injection pressure is fully and effectively applied to the medication unit.

A front seal 112, an interference fit seal 113, a lip seal 114, ensure a gas-tight sealing where necessary.

A location flange 115 ensures a proper positioning of propellant container 23.

In FIG. 20 parts similar to those of above described injector module embodiments are designated with the same reference numbers.

### FIFTH EMBODIMENT OF A DEVICE ACCORDING TO THE INVENTION

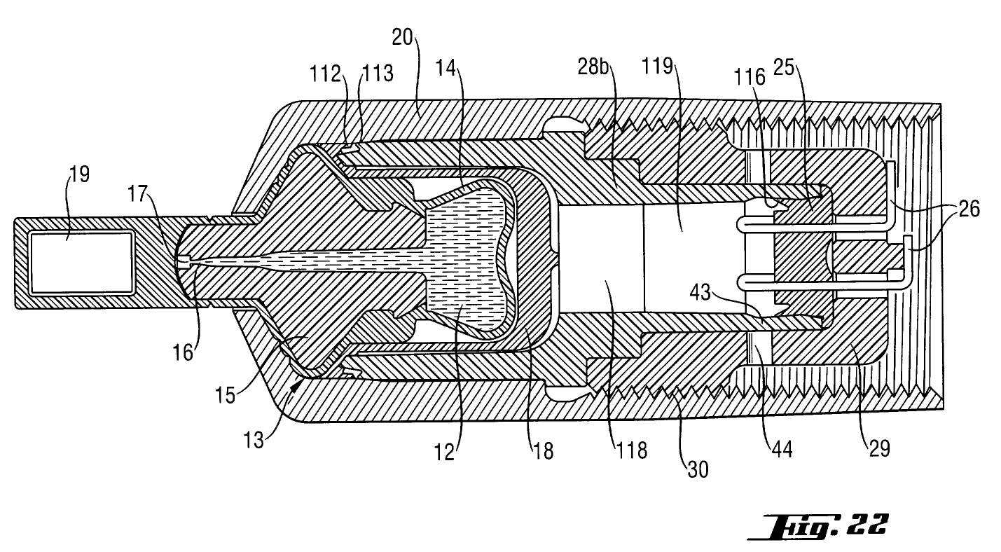

FIG. 21 shows a schematic cross-sectional view of a fifth embodiment of a needleless injector module according to the invention.

This fifth embodiment has components which perform similar functions as in the fourth embodiment, but is characterized by a simplified design that combines the intermediate carrier and the propellant cup into a single part 28a which is e.g. a one-piece part made by molding of a suitable plastic material, e.g. a polyester. This advantageously reduces the number of parts of the injector module and the number of gas-tight seals required. In a preferred embodiment the latter one-piece part is made by injection molding of a polycarbonate.

In a preferred embodiment a liner containing a propellant is lodged in the propellant cup portion of the combined intermediate carrier and propellant cup 28a.

### SIXTH EMBODIMENT OF A DEVICE ACCORDING TO THE INVENTION

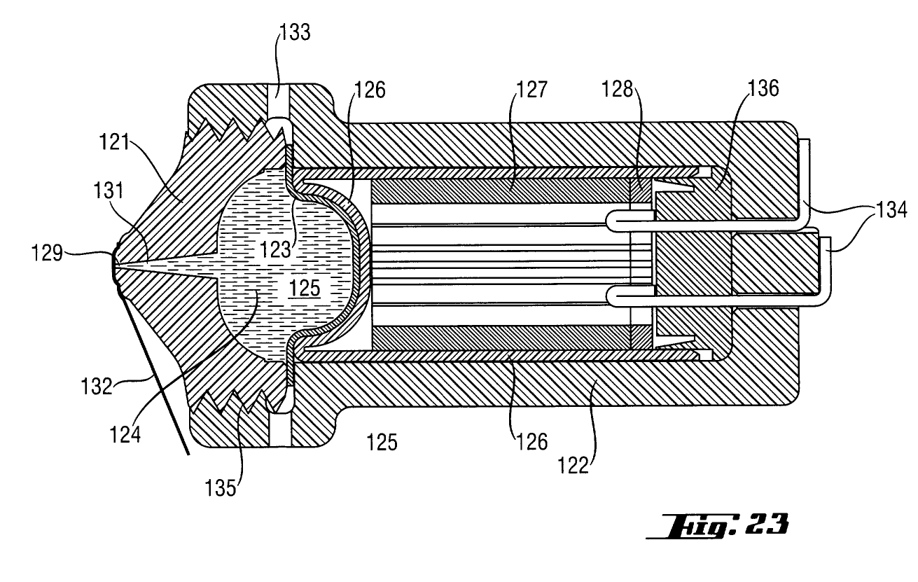

FIG. 22 shows a schematic cross-sectional view of a sixth embodiment of a needleless injector module according to the invention.

In this sixth embodiment, pressure shell 20 is a first rigid housing part which has a zone for receiving the medication unit 13. A rear housing part is a second rigid housing part which is adapted for receiving and/or carrying pyrotechnical means like a propellant and ignition means, e.g. an ignition layer and means for electrically heating the ignition layer. The first and second housing parts are connectable with each other, e.g. by a screw connection 30, and define a single chamber 118 wherein both the medication unit and the propellant are lodged. A deformable barrier 18, e.g. a rubber layer, is arranged within the latter single chamber and divides it in two zones, a first zone wherein said medication unit is located and a second zone 119 where said propellant is located. When the propellant is ignited, the pressure generated by ignition of the propellant is directly applied to deformable barrier 18 and thereby to the flexible wall 14 of medication unit for ejecting the medication contained in reservoir 12 through ejection outlet 16 of nozzle 15 of medication unit 13.

In a preferred embodiment shown by FIG. 22, this sixth embodiment has a one-piece, intermediate carrier 28b which contains a combustion chamber 118. This chamber contains zone 119 wherein a propellant is received and lodged. In a preferred embodiment, the intermediate carrier 28b is made by molding of a plastic material, e.g. by injection molding of a polycarbonate.

In a preferred embodiment a liner containing a propellant is lodged in zone 119.

### SEVENTH EMBODIMENT OF A DEVICE ACCORDING TO THE INVENTION

FIG. 23 shows a schematic cross-sectional view of a sixth embodiment of a needleless injector module according to the invention. This embodiment comprises a nozzle body 121 and a rigid housing 122 made of a plastic material. Housing 122 has a first open end adapted to receive and be connected with the nozzle body 121 and a second closed end.

The interior of the housing 122 defines a chamber which extends between the open end and the closed end of housing 122. That chamber is adapted to receive a first deformable diaphragm 123 which together with a cavity 124 of nozzle body 121 forms a medication chamber 125 suitable for receiving a predetermined amount of a medication, and a second deformable diaphragm 126 a portion of which extends around a portion of the first deformable diaphragm 123. The second deformable diaphragm 126 and the housing 122 form together a chamber for receiving a propellant 127 and means for igniting the propellant 127.

Housing 122 further contains an ignition layer 128 which is in contact with or is an integral part of the one-piece propellant pellet 127 and means for igniting the ignition layer 128. Such means include e.g. ignition pins 134 through which electrical energy is supplied to an electrical resistor used for heating the ignition layer. Ignition pins pass through bores in the closed end of housing 122 and through bores in an ignition plate 136.

Nozzle body 121 has at its outer end an orifice 129 which is the outlet of a channel 131 for loading a liquid medication into medication chamber 125 and for ejecting the medication out of this chamber when a gas pressure generated by ignition of the propellant 127 is applied to the second deformable diaphragm 126 and thereby to the first deformable diaphragm 123.

Nozzle body 121 is made e.g. of polypropylene and the first deformable diaphragm 123 is made e.g. of polyethylene. Both polypropylene and polyethylene are materials suitable and accepted for long term storage of many medications.

In the example described with reference to FIG. 22, the amount of medication stored in medication container 124, 125 is e.g. 200 microliters.

An important characteristic of the injector structure shown by FIG. 23 is that both the medication container and the propellant are actually both contained in a single chamber. This structure minimizes heat losses and that minimizes the amount of energy and thereby the amount of propellant required to generate the gas pressure necessary for performing an injection. In the present example an amount of propellant corresponding to about 20 milligrams of a nitrocellulose based composition was used.

The orifice 129 of the nozzle body 121 is sealed by a removable foil seal 132.

In a preferred embodiment the housing 122 and the nozzle body 121 are connectable to each other by a screw connection 135.

The first deformable 123 diaphragm and nozzle body 121 are clamped together by the screw connection of housing 122 and nozzle body 121.

In another preferred embodiment the housing 122 has venting holes 133 located near to the outer edge of the first deformable diaphragm 123. In operation when propellant 127 is ignited and generates pressure, this pressure is applied to the second deformable diaphragm 126 and this diaphragm pressurizes the first deformable diaphragm and thereby the medication contained in medication container 124, 125 and causes fluid to flow into the nozzle channel 131 and be ejected as a jet through orifice 129. The space between the first diaphragm 123 and the second diaphragm 126 is vented by vents 133 to ensure that pressurized gas cannot enter into contact with the medication volume.

In a further preferred embodiment the housing 122 and the nozzle body 121 are so configured and dimensioned that they can withstand alone the pressure generated by ignition of the propellant 127.

Nozzle body 121 has preferably a tapered outer surface which has its smallest cross-section at the orifice 129 at the outer end of the nozzle body 121.

### EXAMPLE OF PROPELLANT FORMS THAT CAN BE USED WITH ANY OF THE ABOVE DESCRIBED EMBODIMENTS OF A DEVICE ACCORDING TO THE INVENTION

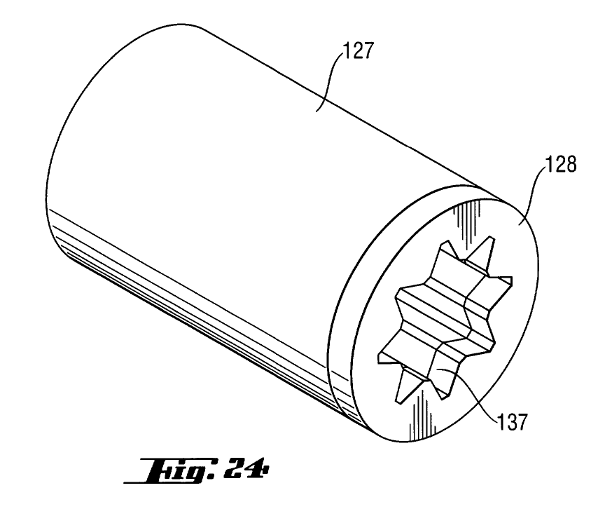

A propellant form which can be used with any of the above described embodiments of a needleless hypodermic injection device is described hereinafter with reference to the above described seventh embodiment of such a device and with reference to FIGS. 23 and 24.

FIG. 23 shows an embodiment, wherein the propellant 127 is a one-piece propellant pellet. This pellet has e.g. the cylindrical or pillar shape shown by FIG. 24 and contains the main propellant charge for making an injection. The specific shape of the pellet can have features which allow to place it at a predetermined position within housing 122, e.g. for ensuring a good contact of the pellet with ignition means.

Within the scope of the invention a propellant pellet is a monolithic structure that contains one or more pre-measured pyrotechnic components. Such a pellet is handled and assembled in the gas generator as a discrete component. Use of such a propellant pellet thus eliminates the need to weigh-out and pour a propellant in powder or liquid form into a propellant container. A preferred embodiment of a propellant pellet of the kind just mentioned has zones having different properties in order to enhance the performance of the pellet. The pellet has e.g. the shape of a cylinder made of a nitrocellulose based composition and one end of this cylinder has an ignition mixture coating and this end of the cylinder is positioned next to an igniter.

Compared with prior art use of propellant in powder form, use of a one-piece propellant pellet offers the advantage of a simplification of the process for manufacturing the injection device, because the pellet comes to the process as a component having a specified weight which is simply inserted into the housing of the injection device, so that no weighing and filling machinery is necessary for handling the pellet. Propellant in powder form has on the contrary to be weighted as part of the manufacturing process and for this purpose weighing and filling machinery is necessary.

Pellets with a wide range of shapes and combinations of material are possible, providing flexibility in tailoring performance and fitting various physical configurations.

In a preferred embodiment, an ignition layer 128 is in contact with or is an integral part of the one-piece propellant pellet 127. Ignition layer 128 preferably contributes to lighting of propellant 127 and additionally provides the energy necessary for generating an initial fast rising pressure pulse.

As shown by FIG. 24, propellant pellet 127 preferably has e.g. a hole 137 which extends through pellet 127 and has a star-shaped cross-section that provides an increased surface area that contributes to a rapid ignition and which provides a gas flow passage through pellet 127.

The following are examples of the chemical and structural composition of a propellant pellet 127:

Example A

Pellet 127 consists of only one grade guncotton which has been processed as a cord and which has a well defined weight per length. Cylindrical pellets 127 having predetermined dimensions and weight are obtained by cutting the cord in equidistant pieces. One end of each pellet so obtained has an ignition mixture coating. Defined positioning of the pellet into a gas generator will bring this coated end of the pellet close to an igniter.

Example B

The base material of a pellet contains a defined mixture of two varieties of guncotton with different fiber length and reactivity. This material is felt and inserted under defined conditions (weight per length/volume) into a thin tube of polyethylene having an inner diameter of e.g. 0.1 mm. Cylindrical pellets 127 having predetermined dimensions and weight are obtained by cutting the tube into adequate cylindrical segments.

Each pellet so obtained is inserted into a gas generator and is arranged close to an igniter.

Example C

A pellet of guncotton according to example A) or example B) is produced by a method wherein additional defined amounts of other materiel like capsules of liquid are included in the pellet.

### Example D

A first pellet of guncotton according to example A) or example B) is produced, but with a shorter length. A second pellet with different properties—with or without propellant properties—is set into a free space within the gas generator after having placed the first pellet within the gas generator.

The second pellet contains e.g. embedded salts, a filler (aerogel) or capsules containing a liquid. The second pellet has a whole in its center (the second pellet has a toroid-like shape) and serves as a modifier of the burning behavior of the first pellet.

A one-piece propellant pellet 127 is so manufactured that the pellet or the method for its manufacture has one or more of the following features in order to achieve desired operation characteristics:

a) Propellant pellet 127 is manufactured from a selected material, e.g. a nitrocellulose based composition, or from a combination of selected materials.

b) Propellant pellet 127 is so manufactured that it has a specified shape and mass.

c) In the process for manufacturing propellant pellets suitable ignition materials can be integrated into the propellant pellet and located at selected spots, inside the pellet or on its outer surface.

d) In the process for manufacturing propellant pellets free space between the pellet and ignition means may be provided by choice of a suitable shape of the pellet. This free space may optionally be filled e.g. with powder or with a filamentary ignition material, e.g. guncotton.

e) The pellet is a mechanical assembly of components with different properties.

f) The pellet includes aggregates of soft filamentary material such as guncotton or capsules of liquid.

g) The pellet includes geometric features such as holes or ribs to increase surface area.

h) A pellet is a structure which fits alone or combined with other pellets properly into the inner space of a gas generator, thus avoiding unduly uncontrolled displacements thereof.

i) A part of the pellet (or an additional pellet) includes a region which only acts as a spacer without propellant properties and which serves for getting the total pellet system properly fitted into the gas generator.

j) The pellet has a self-supporting structure that keeps its shape, e.g. woven, plaited or felted filamentary material structure such as guncotton.

k) The pellet has an additional cover or envelope for stabilizing the structure of the pellet, e.g. a thin tube-like or net-like mantle of e.g. polyethylene or paper-like material.

Two or more one-piece propellant pellets 127 having the same or different characteristics can be arranged within housing 122 with or without intermediate materials between them instead of a single one-piece pellet in order to achieve particular effects like accelerating or delaying certain phases of the combustion of the propellant.

In preferred embodiments, the propellant 127 comprises an array of one-piece propellant pellets having each a predetermined shape, a predetermined chemical composition and a predetermined relative position within the array. Use of one-piece propellant pellets having different chemical compositions and therefore different burning properties make it possible to optimize the variation with time of the injection pressure generated by combustion of the propellant according to predefined criteria. FIGS. 25 to 27 show examples of such arrays.

FIG. 25 shows a stack 141 of cylindrical one-piece pellets 142, 143, 144. In a preferred embodiment, a hole 145 extends through the central portion of stack 141 (shown schematically).

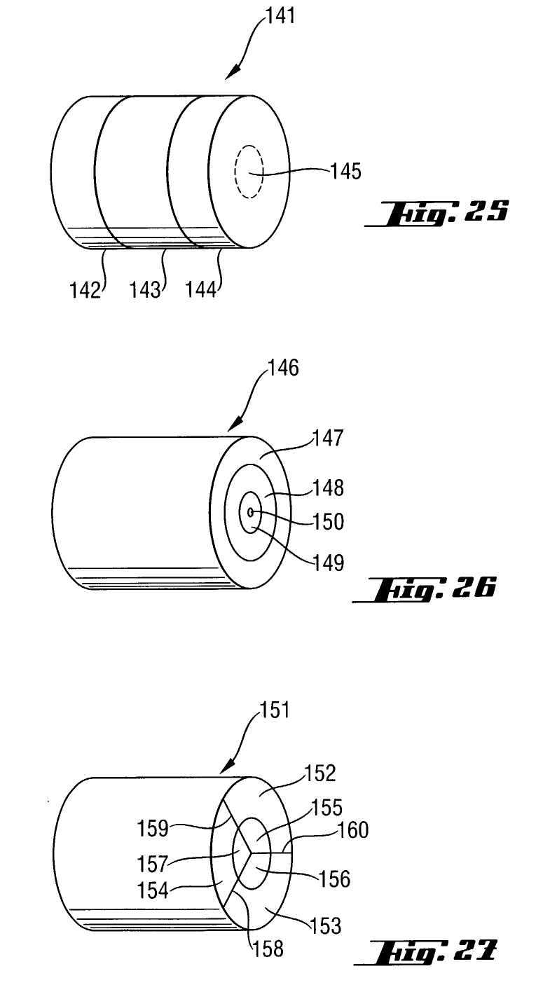

FIG. 26 shows an array 146 of concentric cylindrical one-piece pellets 147, 148, 149. In a preferred embodiment, a hole 150 extends through the central portion of array 146.

FIG. 27 shows an array 151 of one-piece pellets 152 to 157. Pellets 152 to 154 have each the shape of a segment of a cylinder having a predetermined wall thickness. Such segments are obtained by cutting a cylinder along planes parallel to the symmetry axis of the cylinder and passing through radii 158, 159, 160. Pellets 155 to 157 have each the shape of a segment of a rod having a predetermined diameter. Such segments are obtained by cutting a rod along planes parallel to the symmetry axis of the rod and passing through radii 158, 159, 160. In a preferred embodiment, a hole (not shown) extends through the central portion of array 151.

In preferred embodiments of the examples shown by FIGS. 25 to 27, an ignition layer is in contact with or is an integral part of an array of one-piece propellant pellets.

Propellant pellets of the above described types preferably have a coating protecting them against deterioration caused by humidity or by abrasion; in particular abrasion caused by transport, handling or storage processes.

### IGNITION BY MECHANICAL IMPACT

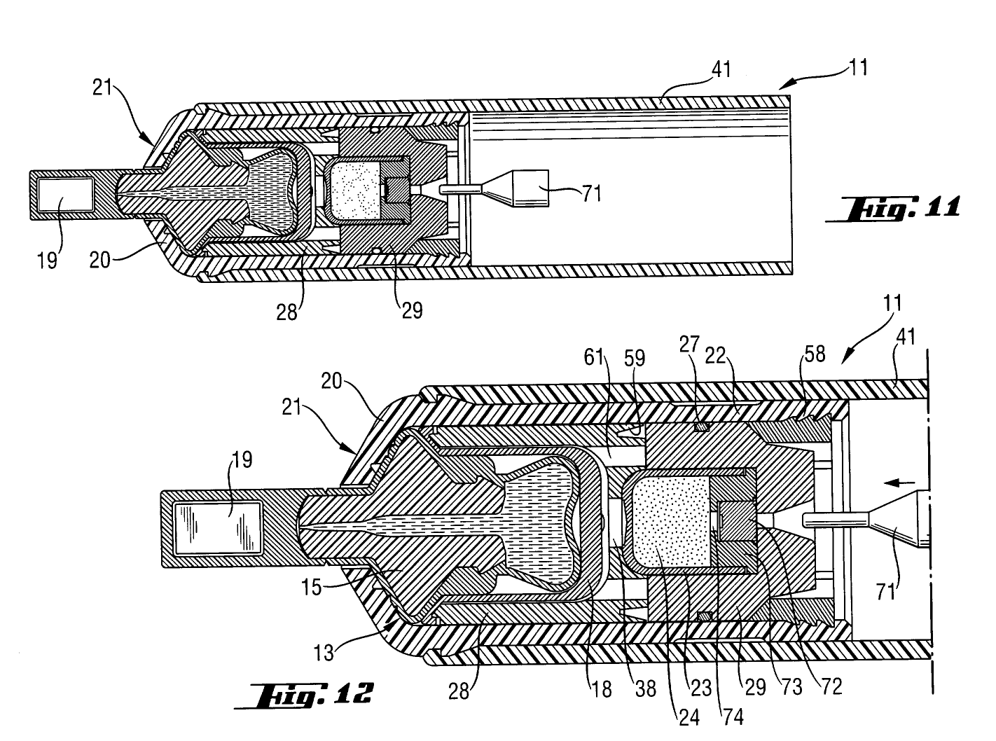

FIG. 11 shows a schematic cross-sectional view of the third embodiment shown by FIG. 10a in combination with mechanical impact ignition means. FIG. 12 shows an enlarged view of an end part of the injector module shown in FIG. 11. The means for ignition by mechanical impact described hereinafter with reference to FIG. 11 and applied to the third embodiment shown by FIG. 10a can also be applied to the above described first and second embodiments of a injector module according to the invention.

The ignition means represented in FIGS. 11 and 12 comprise an impact initiated primer 72 which is hold by a primer support 73 and is adapted to be struck by a firing pin mechanism 71. Primer 72 is so positioned with respect to propellant 24 that the hot products of combustion of primer 72 ignite propellant 24.

In a preferred embodiment primer 72 and the firing pin mechanism are preferably an integral part of injector module 11 and used once and discarded.

In another preferred embodiment, primer 72 is an integral part of injector module 11 and is used once and discarded; whereas firing pin mechanism is part of a removable module and is used more than once.

The firing pin 71 is a mechanical member that incorporates a small diameter cylindrical portion with a rounded end that strikes and indents the metal primer shell. This mechanically initiates the pyrotechnic reaction that in turn ignites propellant 24. For this purpose a flash hole 74 connects primer 72 to propellant 24. Typically pin 71 must strike with a kinetic energy of 0.1 to 0.5 joules to achieve reliable ignition. This energy is provided by a preloaded spring that accelerates the firing pin to strike the primer. Other elements in the firing pin mechanism are a trigger latch to retain the preloaded spring until it is released by the user, and a housing structure to guide the motion of the firing pin and hold the spring, trigger and firing pin in an operable relation to each other and the primer. The mechanism may be either incorporated into a disposable injector module and used once, or built into an actuation device that is attached to the injector module for actuation, and then removed and reused.

### PRESSURE VS. TIME DIAGRAM

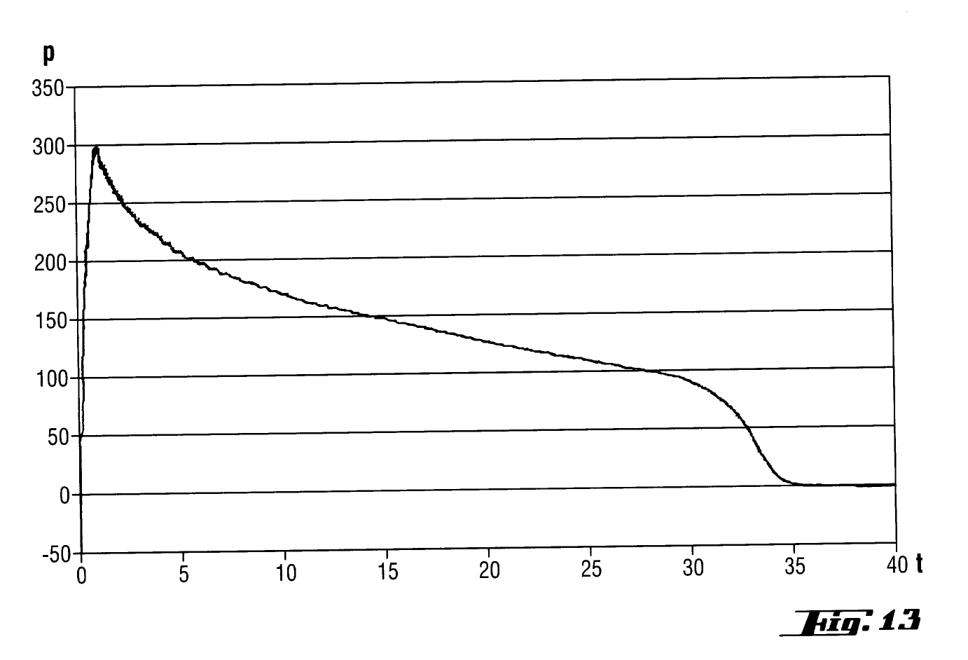

FIG. 13 shows a typical pressure (p, bar) vs. time (t, milliseconds) diagram of the injection pressure exerted on the medication container 13 when an injection is effected with an injector module 11 according to the invention. The pressure values represented are calculated on the basis of corresponding measured force values obtaining by measuring the force exerted by the ejected medication jet on a target. In the diagram of FIG. 13 the instant t=0 is the point of time at which the pressure generated within propellant container 23 by ignition of propellant 24 is large enough to cause rupture of propellant container 23 wail that is in face of elastic barrier 18 and establish a fluidic connection between the interior of propellant container 23 and the chamber containing the elastic barrier 18 and medication unit 13. As represented in FIG. 13 the injection pressure raises very fast, reaches a maximum value of about 300 bar in a very short time interval, a value which is suitable for producing a medication jet that pierces the patient's skin, and then slowly decreases, thereby ensuring that the entire medication volume contained in the medication container is injected.

The pressure vs. time behavior of an injector system according to the invention (represented by the diagram shown by FIG. 13) may be modified in order to modify and adjust the penetration behavior into skin and underlying tissue. This modification is preferably achieved by using a predetermined amount of a basically inert or non-energetic material that is able to exchange heat (heat transfer to and from) with the propellant gas and generate additional gas volume. This material is positioned such that it is contacted by the propellant gas in the second zone 34 of the first chamber after the propellant combustion is complete and the initial peak pressure of about 300 bar has been generated. In one embodiment, the inert material is for example a metal mesh with a defined surface to volume ratio. The initial peak pressure is little affected by the presence of this material since the heat transfer time is short. Following the initial peak pressure the temperatures of the gas and the mesh equilibrate, the mesh being heated and the gas being cooled. This results in a rapid pressure drop. As the gas expands and cools further, the sensible heat stored in the mesh flows back to the gas and sustains the gas temperature and pressure. In a second embodiment non-energetic material undergoes a simple phase change such as the vaporization of a solid or a liquid substance to gas, simultaneously absorbing heat and evolving gas. A solid to solid or solid to liquid phase change without gas evolution is also an option. In a third embodiment the material, e.g. sodium bicarbonate, may undergo a chemical reaction such as the evolution of carbon dioxide from sodium bicarbonate while absorbing heat. In all embodiments pressure is reduced to the extent that temperature is reduced, and increased to the extent that the number of moles of gas is increased.

### ELECTRICAL IGNITION MEANS

When electrical ignition means are used in the above described first, second and third embodiments of an injector module according to the invention such ignition means comprise e.g. an electrically resistive element which is brought in contact with propellant 24. The resistive element is adapted to be heated by a current provided by a source of electrical energy, e.g. a battery. The ignition means further comprise switch contacts for connecting the resistive element to the source of electrical energy.

For ensuring an effective ignition a pyrotechnic ignition material is preferably applied to the electrically resistive element. The pyrotechnic ignition material forms sparks when the resistive element is heated by the current, the sparks causing ignition of the propellant 24.

In a preferred embodiment, the resistive element, the source of electrical energy, and switch contacts are an integral part of the device, and are used only once and discarded.

In another preferred embodiment, the resistive element is an integral part of the single use injection device and is used once and discarded; but the source of electrical energy and the switch contacts are part of a removable module that is used more than once.

### ADDITIONAL SECURITY AND SAFETY FEATURES

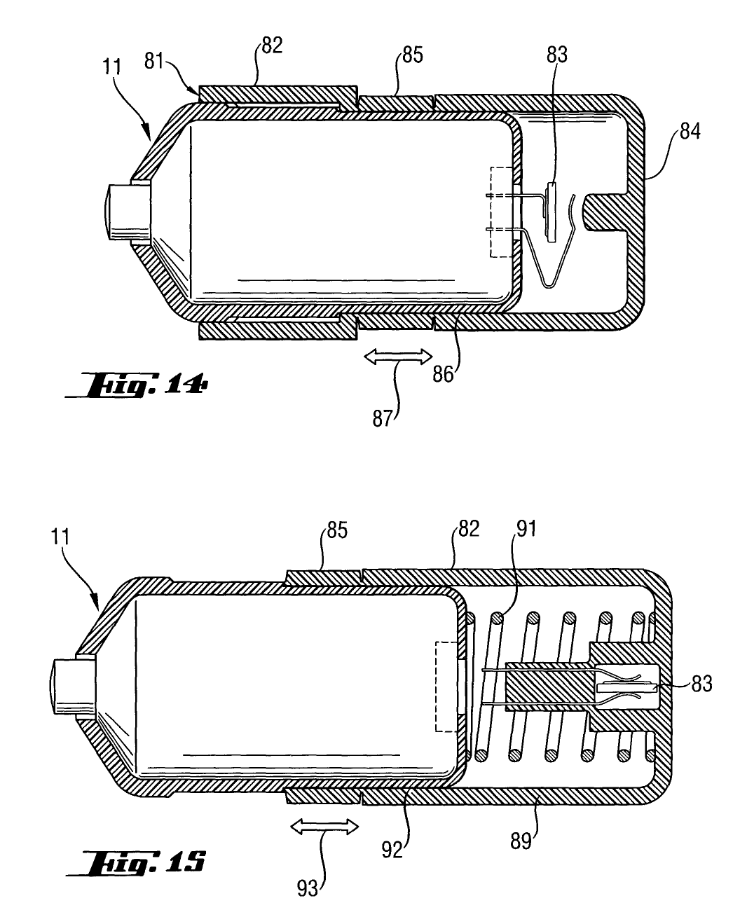

FIG. 14 shows a schematic cross-sectional view of an injector device wherein an injector module 11 according to any of the above described embodiments is arranged within a housing 81 having a grip area 82. This injector device comprises in addition a battery 83 and switch mechanism for ignition. The embodiment shown by FIG. 14 only has an actuation button 84, and does not include any object sensor. Button 84 can be displaced over a range represented by a double head arrow 87 when button 84 is actuated. For this purpose there is a sliding connection 86 between actuation button 84 and module 11. The chance of accidental actuation of the injector device is reduced by a security belt 85 that must be removed before button 84 is pressed. The security means just described are applicable to all above described embodiments of injector module.

FIG. 15 shows a schematic cross-sectional view of an injector device similar to the one shown by FIG. 14 but comprising a switch mechanism for ignition that includes an object sensor mechanism. This object sensor mechanism substantially comprises a slidable housing part 89, a spring 91 arranged as shown and a sliding connection 92 between housing 88 and module 11.

The embodiment of injector device shown by FIG. 15 must be pressed against the injection site for actuation (displacement range). The chance of accidental actuation is reduced by a security belt 85 that must be removed before use. This mechanism is applicable to all above described embodiments.

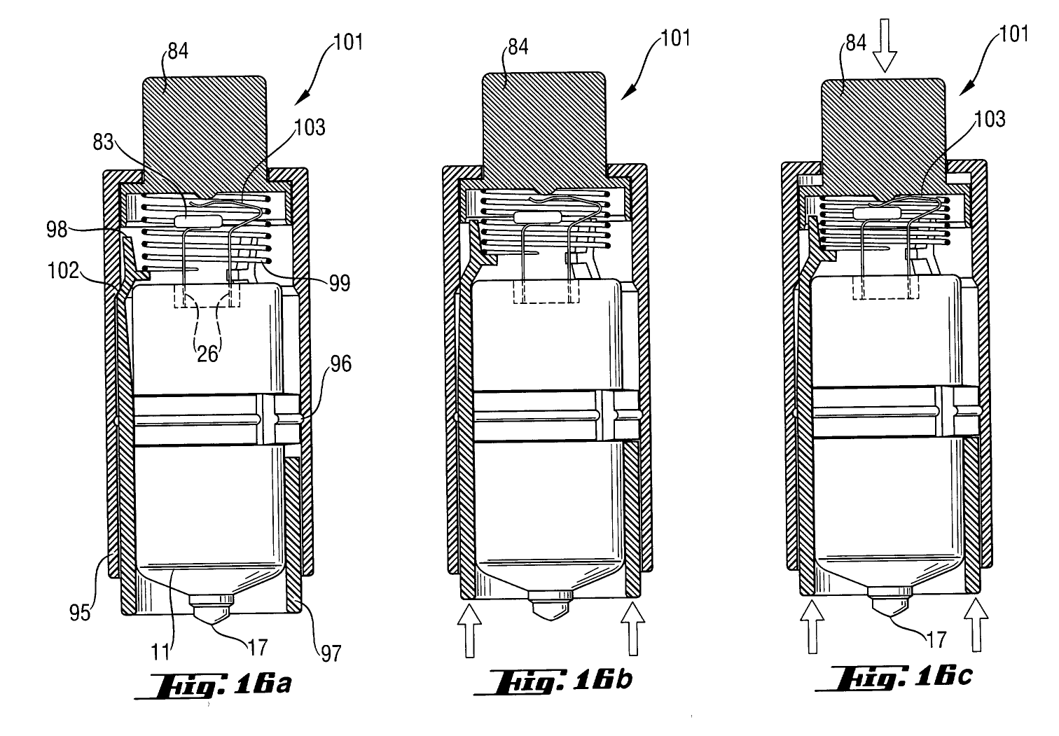

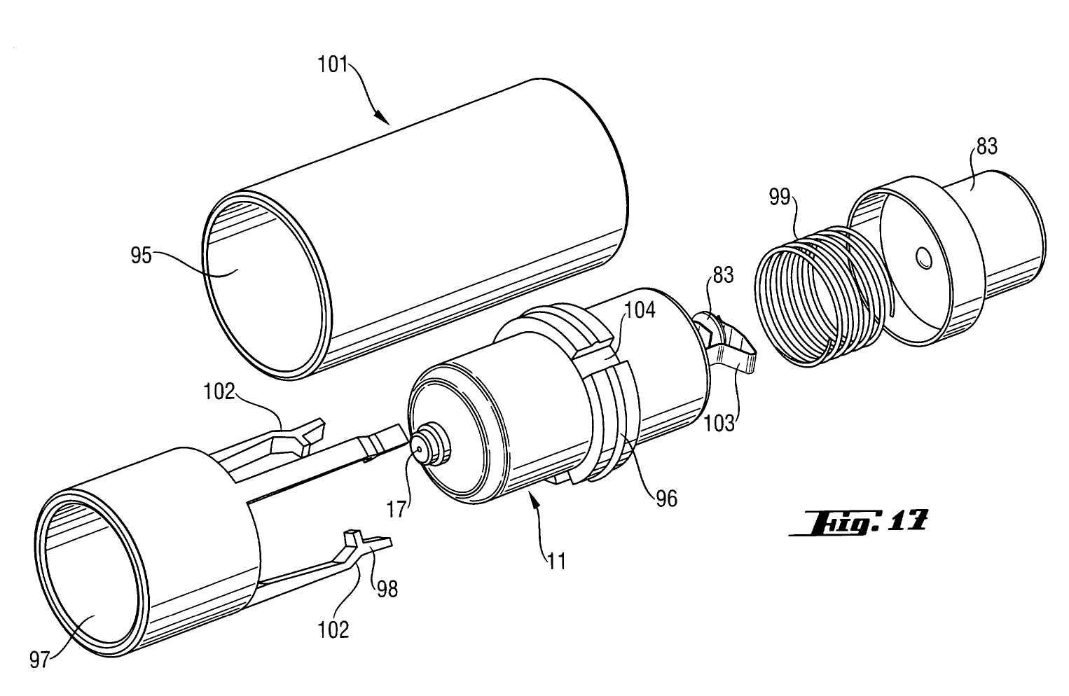

The reliability and security of the operation of an injector device according to the invention is increased by providing it with an interlocking object sensor function as represented in FIGS. 16a, 16b, 16c and FIG. 17.

FIG. 16a is a schematic cross-sectional view of an injector device according to the invention comprising a battery and switch mechanism for ignition that includes an interlocking object sensor function which prevents use of the injector device if certain conditions are not fulfilled. Accidental use of the injector device is thereby prevented.

The provision of such an interlocking object sensor function makes sure that the injector device must first be pressed against the injection site before the actuation button can be pressed. Pressing the button first, and then applying the injector device to the injection site does not work. This mechanism is applicable to all the embodiments described above.

FIG. 16a shows a cross-sectional view of the injector device in a first state before use thereof.

FIG. 16b shows a cross-sectional view of the injector device in a second state as the injector device is pressed against the injection site, the object sensor ring is pushed back, the actuation button being thereby unlocked.

FIG. 16c shows a cross-sectional view of the injector device in a third state with the actuation button in a position at which it closes an ignition switch 103.

FIG. 17 shows a perspective exploded view of components of the object sensor interlock represented in FIGS. 16a to 16c.

The object sensor interlock represented in FIGS. 16a, 16b, 16c and FIG. 17 requires that the injector device according to the invention be pressed against the surface at the injection site before the actuation button is allowed to be moved for carrying out the injection. The purpose of this is to increase the likelihood of a successful medication injection and reduce the chance of accidental actuation resulting in wasted medication or injury, particularly with inexperienced users.

The injector device 101 represented in FIGS. 16a, 16b, 16c comprises an injector module 11 according to any of the embodiments described above which contains the medication, propellant and electrical ignition means.

Injector module 11 is enclosed in a structural housing 95. Two ignition conductors 26 extend from the rear of the injector module 11 and have the shape of flat metal spring members. One is structurally and electrically bonded to one terminal of a battery 83. The other is positioned so that when it is pushed by the actuation button 84 it contacts the other battery terminal. This completes the electrical circuit and enables actuation of the injector device by electrical ignition of the propellant.

Injector module 11 is rigidly connected to surrounding housing 95 through a snap joint 96 on a raised portion of the injector module 11. For performing an injection, the user grips the housing 95 to press the injection nozzle 17 against the injection site on the skin.

An object sensor ring 97 surrounds the nozzle end of the injector module 11 and is slidably mounted in an annular space between the injector module and the surrounding housing 95. The rear part of object sensor ring 97 carries fingers 98 that extend to the rear of the injector device through clearance grooves 104 (shown in FIG. 17) in the raised portion of the injector device 11.

The object sensor ring 97 and fingers 98 are urged forward by a coil spring 99. In this position the ends of the fingers 98 block motion of the actuation button 84 and prevent actuation. The other end of the spring 99 urges the actuation button 84 to the rear.

When the user presses the nozzle 17 against the skin, the object sensor ring 97 contacts the skin around the injection site and is pushed toward the rear of the injector device against the spring force. The fingers 98 are deflected inward by a surface of a cam 102 formed on the housing interior. This unlatches the actuation button 84 so that it can move far enough to push against and actuate a switch for ignition contact and thereby actuate the injector device as shown by FIG. 16c.

There is a sequential logic built into the injector device. The object sensor ring 97 must be pushed in first, and then the actuation button 84 may be pushed. If the actuation button 84 is pushed first it contacts the fingers 98, and prevents actuation by pushing on the object sensor ring 97. Neither the actuation button 84 alone nor the object sensor ring 97 alone is able to actuate the injector device.

### OPTIMIZING THE INJECTION CONDITIONS BY DESIGN OF THE NOZZLE OF THE MEDICATION UNIT

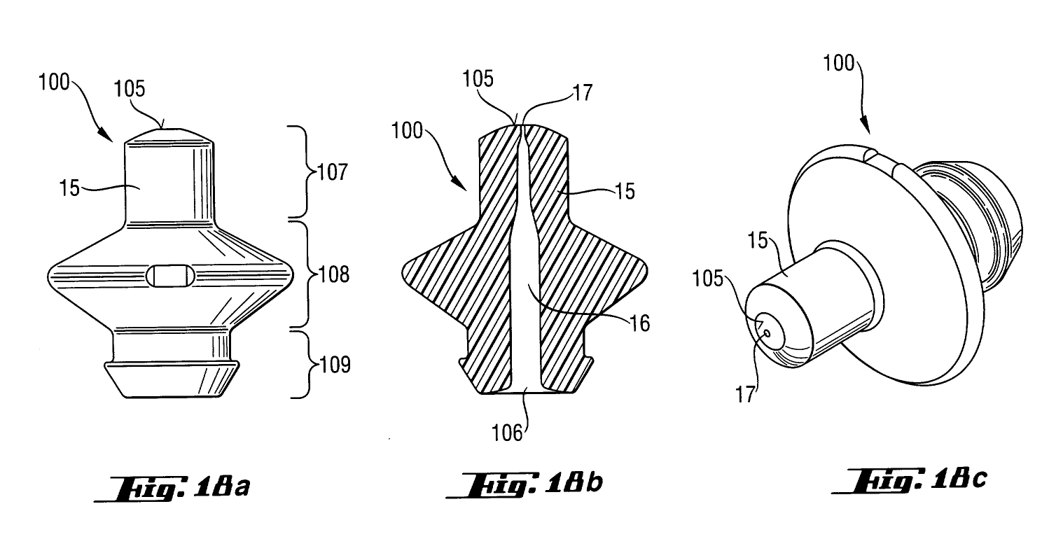

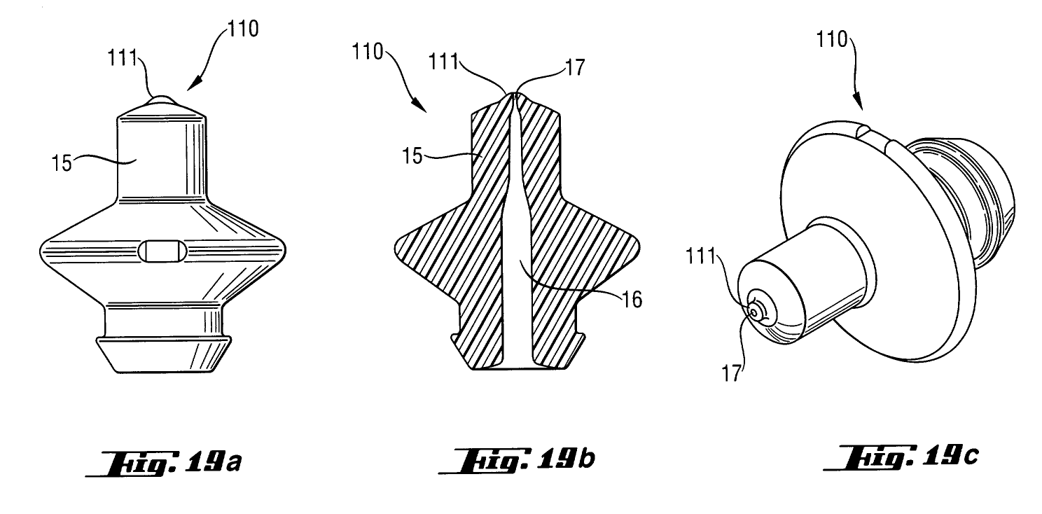

FIGS. 18a to 18c show different views of a first preferred embodiment of a nozzle of the medication unit which is part of any of the above described embodiments of an injector module.

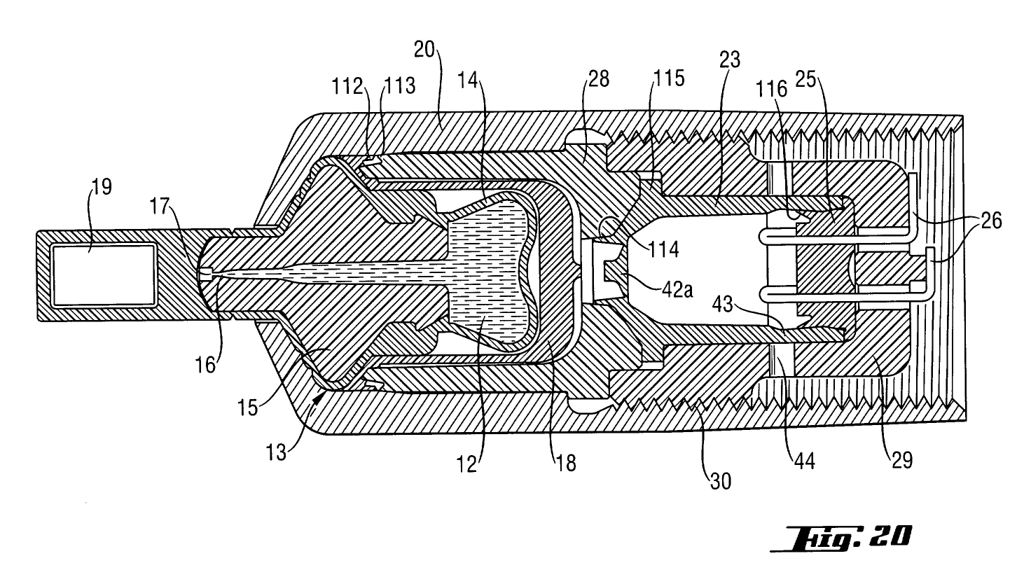

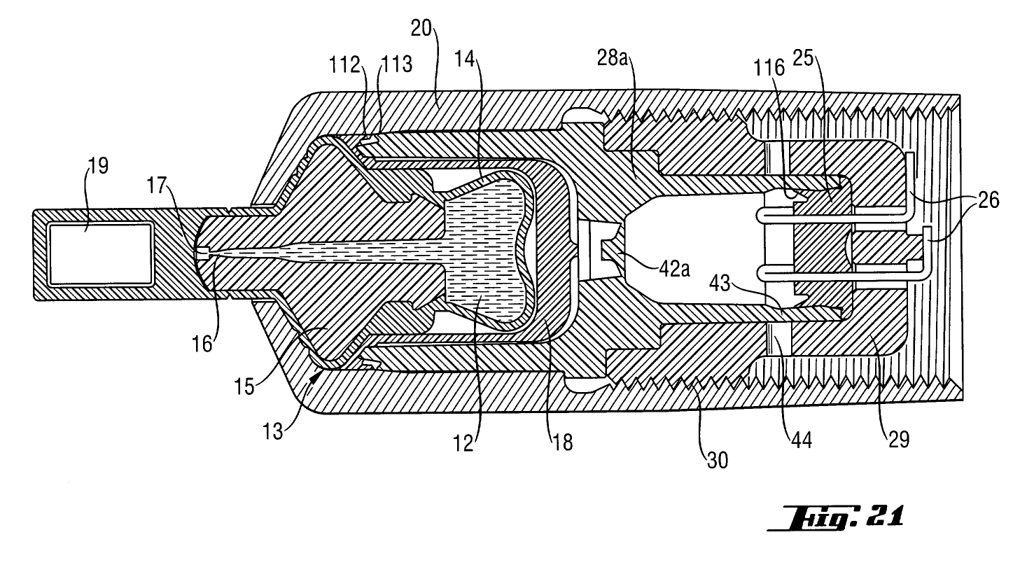

FIGS. 19a to 19c show different views of a second preferred embodiment of a nozzle of the medication unit which is part of any of the above described embodiments of an injector module.

The design of each of these embodiments, which are preferably made of polypropylene, is based on the discovery that the details of the interaction of the liquid medication jet with the skin has an influence on the pressure required to achieve a complete injection.

The nozzle 100 shown in FIGS. 18a to 18c has a flat surface 105 in contact with the skin and the minimum orifice diameter lies in the plane of this surface. This feature ensures that the fluid velocity is at a maximum when it contacts the skin.

The nozzle shown 100 in FIGS. 18a to 18c has a nozzle body 15 which has a longitudinal axis which is also a rotation symmetry axis of the body. The nozzle body comprises an injection channel 16 which has a symmetry axis that coincides with the symmetry axis of the body. The end of the injection channel having a wide opening 106 is connectable to a medication container. The opposite end of the injection channel 16 is an outlet 17 for delivering medication ejected through the injection channel.

The body of the nozzle has a neck portion 107 that ends in a first end which forms a contact surface with the skin at the injection site, a basis portion 109 that ends in a second end opposite to the first end of the body, and an intermediate portion 108 that extends between the neck portion and the basis portion.

The injection channel 16 of the nozzle shown in FIGS. 18a to 18c opens into an orifice 17 located at a flat top 105 of the nozzle. That orifice is in direct contact with the skin at the injection site during an injection.

FIGS. 19a to 19c show a second embodiment of a nozzle 110 in which the surface in contact with the skin is a dome 111 that stretches and tensions the skin. The nozzle shown in FIGS. 19a to 19c differs from the nozzle shown in FIGS. 18a to 18c substantially in that the end of the nozzle body 15 which is in contact with the injection site during an injection has a rounded shape that projects towards the injection site. The minimum orifice diameter is at the peak of the dome in contact with the skin. This ensures that the skin is more easily penetrated by the liquid jet, because it is stretched and in tension.

Although preferred embodiments of the invention have been described using specific terms, such description is for illustrative purposes only, and it is to be understood that changes and variations may be made without departing from the spirit or scope of the following claims.

1. A needleless hypodermic injection device comprising
a housing having a first chamber and a second chamber therewithin, said housing having an opening;
said first chamber comprising a medication unit for storing a preselected volume of a liquid medication, said medication unit having a first region and a second region in fluid communication with each other, said first region being deformable and said second region having an ejection outlet extending outwardly through said opening in said housing, and
said second chamber comprising pyrotechnic means for generating a gas including a propellant container, a predetermined amount of a propellant within said propellant container, and ignition means for selectively igniting said propellant,
said first chamber comprising two zones, a first zone containing said medication unit and a second zone in communication with said second chamber, so that upon ignition of the propellant in the second chamber gas generated thereby expands into said second zone of said first chamber, exerting pressure on, deforming said deformable first region of said medication unit and thereby causing an ejection of said liquid medication through said ejection outlet with sufficient force to deliver the medication through the skin of the patient.

2. A device according to claim 1, wherein said housing has at least one deformable portion that is deformed in the event that the gas pressure within said housing reaches a predetermined value above a preselected injection pressure, and thereby venting said housing and substantially preventing a rupture of the housing.

3. A device according to claim 1, wherein a material for forming said housing comprises a deformable plastic material.

4. A device according to claim 1, wherein a material for forming said housing comprises a metal.

5. A device according to claim 4, wherein said metal for forming said housing is selected from the group consisting of aluminum and steel.

6. A device according to claim 1, being configured so that once said propellant is ignited and said liquid medication is discharged, said device cannot be refilled and rearmed without substantially destroying said device, thereby rendering said device single-use.

7. A device according to claim 1, wherein a wall of said housing has an area of reduced structural strength disposed to cooperate with sealing means adapted to yield at a preselected pressure so as to allow gas escape in a controlled way if a pressure peak above a preselected maximum arises within the housing thereby releasing the pressure.

8. A device according to claim 1, wherein an assembly of said housing and of the components contained therein has at least one predetermined leakage area to provide a release of pressure in the event that the internal pressure reaches a predetermined level above said normal injection pressure, and thereby venting said housing and substantially preventing a rupture of the housing.

9. A device according to claim 1, wherein said housing has a predetermined failure area for allowing the housing to break if a pressure peak above a preselected maximum arises within the housing, thereby allowing gas to escape from the housing in a controlled way.

10. A device according to claim 1, wherein a lateral wall of said propellant container has at least one safety rupture area and that said housing of the propellant container has a corresponding safety vent hole therethrough disposed substantially adjacent said rupture zone.

11. A device according to claim 3, wherein said deformable plastic material for forming said housing is a polycarbonate.

12. A device according to claim 1, wherein said propellant chamber has a wall which before ignition of the propellant serves as a dividing wall between a first cavity in said first chamber and a second cavity in said second chamber.

13. A device according to claim 1, wherein said housing includes a support member having
a first cavity defining a portion of said first chamber,
a second cavity defining a portion of said second chamber, and
a dividing wall separating said first cavity from said second cavity, said wall having an opening therethrough for allowing a flow of gas generated by said pyrotechnic means from one of said cavities to the other,
said support member substantially filling the space between said cavities and said housing.

14. A device according to claim 13, wherein said support member is formed from a rigid, plastic material said material being disposed to break when subjected to sufficient mechanical stress.

15. A device according to claim 14, wherein said rigid plastic material is a polycarbonate.

16. A device according to any of claims 13, wherein a volume of space between said medication unit and said dividing wall of said support member is substantially smaller than the volume of said propellant container.

17. A device according to claim 1, wherein said housing is substantially enveloped by a tubular layer.

18. A device according to claim 17, wherein said tubular layer is formed from a resilient material disposed to form an outer shield to substantially protect the user of the device from exhaust hot gas generated by said pyrotechnic means vented from said housing and from rupture of said housing.

19. A device according to any of claim 18, wherein said resilient material for forming said tubular layer is selected from the group consisting of a polymeric material selected from a polyethylene, and a metallic material selected from a soft steel and a soft aluminum.

20. A device according to any of claims 19, wherein said material selected for said tubular layer is selected from a material that is substantially unaffected by common cleaning and disinfecting liquids.

21. A device according to claim 17 wherein said tubular layer is formed from a two layer structure comprising an outer tubular layer and an inner tubular layer, said outer layer being formed from a tough elastic material that is substantially resistant to common cleaning and disinfecting liquids, and said inner layer being formed from a sponge like, porous layer formed from the same material as the outer layer or from another material.

22. A device according to any of claim 17, wherein said tubular layer has a thickness of about 0.4 millimeter

23. A device according to claim 12, wherein said wall of said propellant container has a zone of reduced thickness disposed between an interior of the propellant container and said first chamber, said zone of reduced thickness being so configured and dimensioned that it bursts when said pyrotechnic means is activated thereby creating an opening between said propellant container and said first chamber when the pressure developed by the gas generated within the propellant container after ignition of the propellant reaches a predetermined value, said predetermined value being lower than a maximum pressure value sufficient to inject the medication.

24. A device according to claim 23, wherein at least an inner wall of said propellant container is formed from a plastic material having a low thermal conductivity so that said propellant container absorbs a substantially no heat from the hot gas generated within container by ignition of propellant, said plastic material being substantially non-chemically reactive with either the propellant or with the hot gas.

25. A device according to claim 24, wherein said plastic material for forming said inner wall of said propellant container is a polyoleofine.

26. A device according to claim 24, wherein said plastic material is a polyethylene.

27. A device according to claim 1, wherein said propellant container comprises limiting means for limiting the amount of propellant that can be introduced into the propellant container.

28. A device according to claim 27, wherein said limiting means is formed from an aerogel.

29. A device according to claim 27, wherein said limiting means is an air filled bag formed from a polyolefinic material.

30. A device according claim 1, wherein said propellant is selected from a gas generating propellant.

31. A device according to claim 30 wherein said gas generating propellant is a nitrocellulose based composition.

32. A device according to claim 1, wherein a barrier formed from an elastic material divides said first zone from said second zone.

33. A device according to claim 32, wherein said material for forming said elastic barrier is reinforced with woven fibers.

34. A device according to claim 33, wherein said woven fibers are polyamide fibers.

35. A device according to claim 34, wherein said woven fibers are polyaramide fibers.

36. A device according to claim 1 wherein said ignition means comprise an electrically resistive element disposed to contact said propellant, said resistive element being adapted to be sufficiently heated to ignite said propellant by a current provided by a source of electrical energy selectively applied through switch means.

37. A device according to claim 36, wherein said ignition means comprises a pyrotechnic ignition material applied to said electrically resistive element, said material thereby chemically reacting and generating heat and hot particles when said resistive element is sufficiently heated by current, said generated heat and hot particles causing ignition of said propellant.

38. A device according to claim 36, wherein said electrically resistive element, said source of electrical energy, and said switch means are an integral part of said device, and substantially cannot be reused once activated.

39. A device according to claim 36, wherein said resistive element is an integral part of said pyrotechnic means; and wherein said source of electrical energy, and switch means are part of a removable module capable of being used more than once.

40. A device according to claim 1, wherein said ignition means comprises an impact initiated primer struck by a firing pin mechanism thereby providing hot combustion products, said primer disposed to said propellant so that the hot products of combustion of said primer ignite said propellant.

41. A device according to claim 40, wherein said primer and said firing pin mechanism are an integral part of said pyrotechnic means and substantially cannot not be replaced and reused once discharged.

42. A device according to claim 40, wherein said primer is an integral part of said pyrotechnic means and substantially cannot be replaced and reused once discharged; and wherein said firing pin mechanism is part of a removable module capable of being used more than once.

43. A device according to claim 1, wherein said ignition means comprises an interlock, said interlock being opened to enable an actuation of said ignition means by an outer end of said injection outlet being pressed against the patient's skin with a predetermined force sufficient to open said interlock thereby enabling selective actuation of said ignition means by the user.

44. A device according to claim 43, wherein said interlock comprises a sliding ring substantially surrounding said outer end of said injection outlet, and, said sliding ring being biased toward said outer end, so that when said sliding ring is pressed against the patient's skin with said predetermined force, said interlock is opened.

45. A device according to claim 1, wherein said housing has a propellant chamber vent passage therethrough so that gas generated within said housing is vented to atmosphere.

46. A device according to claim 45, wherein said propellant chamber vent passage has a preselected flow resistance so that a gas flow through said vent passage after said pyrotechnic means is activated is substantially negligible during the injection period, said gas being substantially vented to atmospheric pressure after said injection period.

47. A device according to claim 45, wherein said propellant chamber vent passage includes a flow resistance element such that gas flow through said passage is substantially negligible during an injection period of about 50 milliseconds, said flow resistance element permitting a gas venting of the device through said propellant chamber vent passage to atmospheric pressure after about 10 seconds.

48. A device according to claim 47, wherein said flow resistance element for said propellant chamber vent passage comprises a temperature sensitive substance for substantially blocking flow during the 50 millisecond injection period, and said temperature sensitive substance being subsequently melted from propellant heat thereby substantially reducing gas flow resistance in said passage and venting the device to atmospheric pressure.

49. A device according to claim 48, wherein said temperature sensitive substance is a wax with a sharply defined melting point.

50. A device according to claim 47, wherein said flow resistance device comprises a gasket based on a cellulose or a paper inserted in said passage thereby forming a controlled leak for venting the housing after the injection period.

51. A device according to claim 1, wherein said second zone of said first chamber of said housing contains therewithin a predetermined amount of a substantially non-energetic material for interacting with hot gas generated by ignition of the propellant, and thereby modulating temperature and pressure of the hot gas in said second zone.

52. A device according to claim 51, wherein said interaction is a thermal interaction of said non-energetic material with said hot gas.

53. A device according to claim 52, wherein said interaction between said material with said hot gas produces additional gas.

54. The device of claim 1 wherein said ejection outlet extending outwardly through said opening in said housing comprises a nozzle,
said nozzle having a body having a longitudinal axis, said longitudinal axis being a rotation symmetry axis of said nozzle body,
said nozzle body comprising an injection channel having a symmetry axis coincident with said symmetry axis of said nozzle body,
said injection channel having at a first end thereof an inlet connectable to a medication container, said injection channel having at a second end thereof opposite to said first end an outlet for delivering medication ejected through said injection channel, and
said body having a neck portion projecting outwardly said opening in said housing that ends in a first end which serves as a contact surface with the patient's skin at the injection site, a basis portion that ends in a second end opposite to said first end of said body, and an intermediate portion that extends between said neck portion and said basis portion.

55. A nozzle according to claim 54 being formed from polypropylene.

56. A nozzle according to claim 54 wherein said injection channel opens into an orifice located at a flat top of the nozzle, said orifice being disposed to be in direct contact with the skin of the patient at the injection site during an injection.

57. A nozzle according to claim 54, wherein the first end of the nozzle body has a rounded shape that projecting outwardly towards the injection site.

58. A device according to claim 13, wherein said support member and said propellant container are integrally formed as a single article of manufacture.

59. A device according to claim 58, wherein said support member and said propellant container are integrally formed by forming from a plastic material.

60. A device according to claim 59, wherein a liner comprising a propellant is disposed within said propellant container.

61. A needleless hypodermic injection device for injecting a liquid medication comprising
a rigid housing,
said housing having therewithin a nozzle body defining a cavity and projecting outwardly from a first open end adapted to receive and be connected with the nozzle body and a second closed end,
said housing having an interior defining a chamber extending between said open end and said closed end said chamber being adapted to receive
a first deformable diaphragm which together with said cavity of said nozzle body forming a medication chamber suitable for receiving a predetermined amount of a medication, and
a second deformable diaphragm at least a portion of which extends around a portion of said first deformable diaphragm, said second deformable diaphragm and said housing thereby forming together a chamber for receiving pyrotechnic means for generating gas and ignition means for igniting said pyrotechnic means,
said nozzle body having at its outer end an orifice which is the outlet of a channel for loading a liquid medication into the medication chamber and for ejecting said medication out of said chamber when a gas pressure generated by an ignition of said pyrotechnic means is applied to said second deformable diaphragm and thereby to said first deformable diaphragm.

62. A device according to claim 61, wherein the orifice of the nozzle body is sealed by a removable foil seal.

63. A device according to claim 61, wherein said housing and said nozzle body are connectable to each other by a screw connection.

64. A device according to claim 61, wherein said housing has venting means located near to the outer edge of said first deformable diaphragm.

65. A device according to claim 61, wherein said housing and said nozzle body are configured and dimensioned to withstand the pressure generated by ignition of the propellant.

66. A device according to claim 61, wherein said nozzle body has a tapered outer surface which has its smallest cross-section at the orifice disposed at the outer end of the nozzle body.

67. A device for performing a needleless hypodermic injection of a liquid medication contained in a medication unit within the device, said device including pyrotechnical means for generating sufficient gas within the device to provide a predetermined pressure value necessary for injecting the medication, said device comprising
(a) a medication unit for storing a volume of liquid medication to be injected, said medication unit having a first region and a second region that are in liquid communication with each other, said first region being deformable and said second region having an ejection outlet,
(b) a first rigid housing part having a first zone for receiving said medication unit,
(c) a second rigid housing part comprising pyrotechnical means comprising a propellant,
said first and second housing parts being connectable with each other and defining a single chamber, and
(d) a deformable barrier arranged within said single chamber for dividing said chamber in two zones, a first zone wherein said medication unit is located and a second zone where said propellant is located,
a gas pressure generated by a selective ignition of said propellant being directly applied to said deformable barrier and thereby to said deformable region of said medication unit for ejecting said medication through said ejection outlet of said second region of said medication unit.

68. A device according to claim 1, wherein said propellant comprises at least one propellant pellet.

69. A device according to claim 68, wherein said propellant comprises an ignition layer.

70. A device according to claim 1, wherein said propellant comprises an array of propellant pellets each having a predetermined shape, a predetermined chemical composition and a predetermined relative position within the array.

71. A device according to claim 70, wherein said propellant comprises an ignition layer.
